SEQUENCE LISTING (24 sequences)

SEQ ID NO: 1 - protein, 115 aa, Amino Acid
  Variable heavy region
QVQLVQSGAEVKKPGASVKVSCKASGYTFTSYYINWVRQAPGQGLEWMGYINPYNGNTRYAQKFQGRVTMTRDTSISTAYMELSRLRSEDTAVYYCARGMLFAHWGQGTLVTVSS

SEQ ID NO: 2 - protein, 110 aa, Amino Acid
  Variable light region
DIVLTQSPSSLSLSPGERATLSCRASQRVSFNYLAWYQQKPGQAPRLLIYGASKRATGIPARFSGSGSGTDFTLTISSLEPEDFAVYYCMQYLSTPRTFGQGTKVEIKRT

SEQ ID NO: 3 - protein, 119 aa, Amino Acid
  Variable heavy region
EVQLLESGGGLVQPGGSLRLSCAASGFTFRGYYMSWVRQAPGKGLEWVSSISGFSSNTYYADSVKGRFTISRDNSKNTLYLQMNSLRAEDTAVYYCARVRYLAYAFDYWGQGTLVTVSS

SEQ ID NO: 4 - protein, 108 aa, Amino Acid
  Variable light region
DIELTQPPSVSVSPGQTASITCSGDSLPDKRAYWYQQKPGQAPVLVIYGDSHRPSGIPERFSGSNSGNTATLTISGTQAEDEADYYCSSWGSRTWVFGGGTKLTVLGQ

SEQ ID NO: 5 - protein, 118 aa, Amino Acid
  Variable heavy region
QVQLVQSGAEVKKPGSSVKVSCKASGGTFSGYYISWVRQAPGQGLEWMGGIIPISGRANYAQKFQGRVTITADESTSTAYMELSSLRSEDTAVYYCARSRSYYHFDLWGQGTLVTVSS

SEQ ID NO: 6 - protein, 112 aa, Amino Acid
  Variable light region
DIALTQPASVSGSPGQSITISCTGTSSDVGRYNSVSWYQQHPGKAPKLMIYRVSKRPSGVSNRFSGSKSGNTASLTISGLQAEDEADYYCQSWASLSNVVFGGGTKLTVLGQ

SEQ ID NO: 7 - protein, 118 aa, Amino Acid
  Variable heavy region
EVQLVQSGAEVKKPGESLKISCKGSGYSFTGYVIHWVRQMPGKGLEWMGRIDPSKSYTRYSPSFQGQVTISADKSISTAYLQWSSLKASDTAMYYCARGLYSGYFDIWGQGTLVTVSS

SEQ ID NO: 8 - protein, 110 aa, Artificial Sequence
  variable light region
DIELTQPPSVSVSPGQTASITCSGDALGSKYVHWYQQKPGQAPVLVIYAKNNRPSGIPERFSGSNSGNTATLTISGTQAEDEADYYCQSRASGIFGRVFGGGTKLTVLGQ

SEQ ID NO: 9 - protein, 688 aa, Artificial Sequence
  Fd chain and tags Artificial Human Antibody comprising Heavy Chain Variable Region; part of Heavy Chain Constant Region; Dimerization Domain sequence  FLAG, and HiS6 Tags
QVQLVQSGAEVKKPGASVKVSCKASGYTFTSYYINWVRQAPGQGLEWMGYINPYNGNTRYAQKFQGRVTMTRDTSISTAYMELSRLRSEDTAVYYCARGMLFAHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSEFKAEMPVLENRAAQGDITTPGGARRLTGDQTAALRDSLSDKPAKNIILLIGDGMGDSEITAARNYAEGAGGFFKGIDALPLTGQYTHYALNRKTGKPDYVTSSAASATAWSTGVKTYNGALGVDIHEKDHPTILEMAKAAGLATGNVSTAELQDATPAALVAHVTSRKCYGPSATSEKCPGNALEKGGKGSITEQLLNARADVTLGGGAKTFAETATAGEWQGKTLREQAQARGYQLVSDAASLNSVTEANQQKPLLGLFADGNMPVRWLGPKATYHGNIDKPAVTCTPNPQRNDSVPTLAQMTDKAIELLSKNEKGFFLQVEGASIDKQDHAANPCGQIGETVDLDEAVQRALEFAKKEGNTLVIVTADHAHASQIVAPDTKAPGLTQALNTKDGAVMVMSYGNSEEDSQEHTGSQLRIAAYGPHAANVVGLTDQTDLFYTMKAALGLKGAPDYKDDDDKGAPHHHHHH

SEQ ID NO: 10 - protein, 215 aa, Artificial Sequence
  Amino acid sequence of light chain Artificial Human Antibody comprising Light Chain Variable Region and Light Chain Constant Region
DIVLTQSPSSLSLSPGERATLSCRASQRVSFNYLAWYQQKPGQAPRLLIYGASKRATGIPARFSGSGSGTDFTLTISSLEPEDFAVYYCMQYLSTPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEA

SEQ ID NO: 11 - DNA, 2064 bp, Artificial Sequence
  DNA sequences of Fd chain with tags
caggtgcaattggtgcagagcggtgcggaagtgaaaaaaccgggtgccagcgtgaaagttagctgcaaagcgtccggatataccttcacttcttactacatcaactgggtgcgccaggccccgggccagggcctcgagtggatgggctacatcaacccgtacaacggcaacacgcgttacgcgcagaaatttcagggccgggtgaccatgacccgtgataccagcattagcaccgcgtatatggaactgagccgtctgcgtagcgaagatacggccgtgtattattgcgcgcgtggtatgctgttcgctcattggggccaaggcaccctggtgactgttagctcagcgtcgaccaaaggcccgagcgtgtttccgctggccccgagcagcaaaagcaccagcggcggcaccgccgcactgggctgcctggtgaaagattatttcccggaaccagtgaccgtgagctggaacagcggtgccctgaccagcggcgtgcatacctttccggcggtgctgcaaagcagcggcctgtatagcctgagcagcgttgtgaccgtgccgagcagcagcctgggcacccagacctatatttgcaacgtcaaccataaaccgagcaacaccaaagtcgataaaaaagtcgaaccgaaaagcgaattcaaggctgaaatgcctgttctggaaaaccgggctgctcagggcgatattactacacccggcggtgctcgccgtttaacgggtgatcagactgccgctctgcgtgattctcttagcgataaacctgcaaaaaatattattttgctgattggcgatgggatgggggactcggaaattactgccgcacgtaattatgccgaaggtgcgggcggcttttttaaaggtatagatgccttaccgcttaccgggcaatacactcactatgcgctgaatagaaaaaccggcaaaccggactacgtcaccagctcggctgcatcagcaaccgcctggtcaaccggtgtcaaaacctataacggcgcgctgggcgtcgatattcacgaaaaagatcacccaacgattctggaaatggcaaaagccgcaggtctggcgaccggtaacgtttctaccgcagagttgcaggatgccacgcccgctgcgctggtggcacatgtgacctcgcgcaaatgctacggtccgagcgcgaccagtgaaaaatgtccgggtaacgctctggaaaaaggcggaaaaggatcgattaccgaacagctgcttaacgctcgtgccgacgttacgcttggcggcggcgcaaaaacctttgctgaaacggcaaccgctggtgaatggcagggaaaaacgctgcgtgaacaggcacaggcgcgtggttatcagttggtgagcgatgctgcctcactgaactcggtgacggaagcgaatcagcaaaaacccctgcttggcctgtttgctgacggcaatatgccagtgcgctggctaggaccgaaagcaacgtaccatggcaatatcgataagcccgcagtcacctgtacgccaaatccgcaacgtaatgacagtgtaccaaccctggcgcagatgaccgacaaagccattgaattgttgagtaaaaatgagaaaggctttttcctgcaagttgaaggtgcgtcaatcgataaacaggatcatgctgcgaatccttgtgggcaaattggcgagacggtcgatctcgatgaagccgtacaacgggcgctggagttcgctaaaaaggagggtaacacgctggtcatagtcaccgctgatcacgcccacgccagccagattgttgcgccggataccaaagctccgggcctcacccaggcgctaaataccaaagatggcgcagtgatggtgatgagttacgggaactccgaagaggattcacaagaacataccggcagtcagttgcgtattgcggcgtatggcccgcatgccgccaatgttgttggactgaccgaccagaccgatctcttctacaccatgaaagccgctctggggctgaaaggcgcgccggactataaagatgacgatgacaaaggcgcgccgcaccatcatcaccatcac

SEQ ID NO: 12 - DNA, 645 bp, Artificial Sequence
  DNA sequence of Light chain Artificial Human Antibody comprising DNA Sequence of Light Chain Variable Region and Light Chain Constant Region
gatatcgtgctgacccagagcccgagcagcctgagcctgagcccgggtgaacgtgccaccctgagctgcagagcgagccagcgtgtttctttcaactacctggcttggtaccagcagaaaccgggccaggccccgcgtctattaatctacggtgcttctaaacgtgcgaccggcattccggcgcgttttagcggcagcggatccggcaccgatttcaccctgaccattagcagcctggaaccggaagactttgcggtgtattattgcatgcagtacctgtctactccgcgtacctttggccagggcacgaaagttgaaattaaacgtacggtggccgcaccgagcgtgtttatctttccgccgagcgatgaacagctgaaaagcggcaccgccagcgtggtgtgcctgctgaacaacttttatccgcgcgaagccaaagtgcagtggaaagtggataacgccctgcaaagcggcaacagccaggaaagcgttaccgaacaggatagcaaagatagcacctacagcctgagcagcaccctgaccctgagcaaagccgattatgaaaaacataaagtgtatgcctgcgaagtgacccatcagggcctgagcagcccagtgaccaaaagttttaaccgcggcgaggcc

SEQ ID NO: 13 - protein, 692 aa, Artificial Sequence
  Fd chain and tags Artificial Human Antibody comprising Heavy Chain Variable Region; part of Heavy Chain Constant Region; Dimerization Domain Sequence (AP) Flag and HiS6 tags
EVQLLESGGGLVQPGGSLRLSCAASGFTFRGYYMSWVRQAPGKGLEWVSSISGFSSNTYYADSVKGRFTISRDNSKNTLYLQMNSLRAEDTAVYYCARVRYLAYAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSEFKAEMPVLENRAAQGDITTPGGARRLTGDQTAALRDSLSDKPAKNIILLIGDGMGDSEITAARNYAEGAGGFFKGIDALPLTGQYTHYALNRKTGKPDYVTSSAASATAWSTGVKTYNGALGVDIHEKDHPTILEMAKAAGLATGNVSTAELQDATPAALVAHVTSRKCYGPSATSEKCPGNALEKGGKGSITEQLLNARADVTLGGGAKTFAETATAGEWQGKTLREQAQARGYQLVSDAASLNSVTEANQQKPLLGLFADGNMPVRWLGPKATYHGNIDKPAVTCTPNPQRNDSVPTLAQMTDKAIELLSKNEKGFFLQVEGASIDKQDHAANPCGQIGETVDLDEAVQRALEFAKKEGNTLVIVTADHAHASQIVAPDTKAPGLTQALNTKDGAVMVMSYGNSEEDSQEHTGSQLRIAAYGPHAANVVGLTDQTDLFYTMKAALGLKGAPDYKDDDDKGAPHHHHHH

SEQ ID NO: 14 - protein, 211 aa, Artificial Sequence
  Amino Acid Sequence of Light Chain Artificial Human Antibody comprising Light Chain Variable Region and Light Chain Constant Region
DIELTQPPSVSVSPGQTASITCSGDSLPDKRAYWYQQKPGQAPVLVIYGDSHRPSGIPERFSGSNSGNTATLTISGTQAEDEADYYCSSWGSRTWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTEA

SEQ ID NO: 15 - DNA, 2076 bp, Artificial Sequence
  DNA Sequences of Fd chain with tags
gaagtgcaattgctggaaagcggcggtggcctggtgcagccgggtggcagcctgcgtctgagctgcgcggcgtccggattcacctttcgtggttactacatgtcttgggtgcgccaggccccgggcaaaggtctcgagtgggtttcctctatctctggtttctcttctaacacctactatgcggatagcgtgaaaggccgctttaccatcagccgcgataattcgaaaaacaccctgtatctgcaaatgaacagcctgcgtgcggaagatacggccgtgtattattgcgcgcgtgttcgttacctggcttacgctttcgattactggggccaaggcaccctggtgactgttagctcagcgtcgaccaaaggcccgagcgtgtttccgctggccccgagcagcaaaagcaccagcggcggcaccgccgcactgggctgcctggtgaaagattatttcccggaaccagtgaccgtgagctggaacagcggtgccctgaccagcggcgtgcatacctttccggcggtgctgcaaagcagcggcctgtatagcctgagcagcgttgtgaccgtgccgagcagcagcctgggcacccagacctatatttgcaacgtcaaccataaaccgagcaacaccaaagtcgataaaaaagtcgaaccgaaaagcgaattcaaggctgaaatgcctgttctggaaaaccgggctgctcagggcgatattactacacccggcggtgctcgccgtttaacgggtgatcagactgccgctctgcgtgattctcttagcgataaacctgcaaaaaatattattttgctgattggcgatgggatgggggactcggaaattactgccgcacgtaattatgccgaaggtgcgggcggcttttttaaaggtatagatgccttaccgcttaccgggcaatacactcactatgcgctgaatagaaaaaccggcaaaccggactacgtcaccagctcggctgcatcagcaaccgcctggtcaaccggtgtcaaaacctataacggcgcgctgggcgtcgatattcacgaaaaagatcacccaacgattctggaaatggcaaaagccgcaggtctggcgaccggtaacgtttctaccgcagagttgcaggatgccacgcccgctgcgctggtggcacatgtgacctcgcgcaaatgctacggtccgagcgcgaccagtgaaaaatgtccgggtaacgctctggaaaaaggcggaaaaggatcgattaccgaacagctgcttaacgctcgtgccgacgttacgcttggcggcggcgcaaaaacctttgctgaaacggcaaccgctggtgaatggcagggaaaaacgctgcgtgaacaggcacaggcgcgtggttatcagttggtgagcgatgctgcctcactgaactcggtgacggaagcgaatcagcaaaaacccctgcttggcctgtttgctgacggcaatatgccagtgcgctggctaggaccgaaagcaacgtaccatggcaatatcgataagcccgcagtcacctgtacgccaaatccgcaacgtaatgacagtgtaccaaccctggcgcagatgaccgacaaagccattgaattgttgagtaaaaatgagaaaggctttttcctgcaagttgaaggtgcgtcaatcgataaacaggatcatgctgcgaatccttgtgggcaaattggcgagacggtcgatctcgatgaagccgtacaacgggcgctggagttcgctaaaaaggagggtaacacgctggtcatagtcaccgctgatcacgcccacgccagccagattgttgcgccggataccaaagctccgggcctcacccaggcgctaaataccaaagatggcgcagtgatggtgatgagttacgggaactccgaagaggattcacaagaacataccggcagtcagttgcgtattgcggcgtatggcccgcatgccgccaatgttgttggactgaccgaccagaccgatctcttctacaccatgaaagccgctctggggctgaaaggcgcgccggactataaagatgacgatgacaaaggcgcgccgcaccatcatcaccatcac

SEQ ID NO: 16 - DNA, 633 bp, Artificial Sequence
  DNA sequence of Light chain Artificial Human Antibody comprising Light Chain Variable Region and Light Chain Constant Region
gatatcgaactgacccagccgccgagcgtgagcgtgagcccgggccagaccgcgagcattacctgtagcggcgattctctgccggacaaacgtgcttactggtaccagcagaaaccgggccaggcgccggtgctggtgatctacggtgactctcatcgtccgagcggcatcccggaacgttttagcggatccaacagcggcaacaccgcgaccctgaccattagcggcacccaggcggaagacgaagcggattattactgctcttcttggggttctcgtacttgggtgtttggcggcggcacgaagttaaccgttcttggccagccgaaagccgccccaagcgtgaccctgtttccgccgagcagcgaagaactgcaagccaacaaagccaccctggtttgcctgatcagcgatttttatccgggtgccgtgaccgtggcctggaaagccgatagcagcccggtgaaagccggcgtggaaaccaccaccccgagcaaacagagcaacaacaaatatgccgccagcagctatctgagcctgaccccggaacagtggaaaagccatcgcagctatagttgtcaagtgacccatgaaggcagcaccgtggaaaaaaccgtggccccgaccgaggcc

SEQ ID NO: 17 - protein, 691 aa, Artificial Sequence
  Fd chain and tags Artificial Human Antibody comprising Heavy Chain Variable Region; part of Heavy Chain Constant Region; Dimerization Domain Sequence (AP) Flag and HiS6 tags
QVQLVQSGAEVKKPGSSVKVSCKASGGTFSGYYISWVRQAPGQGLEWMGGIIPISGRANYAQKFQGRVTITADESTSTAYMELSSLRSEDTAVYYCARSRSYYHFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSEFKAEMPVLENRAAQGDITTPGGARRLTGDQTAALRDSLSDKPAKNIILLIGDGMGDSEITAARNYAEGAGGFFKGIDALPLTGQYTHYALNRKTGKPDYVTSSAASATAWSTGVKTYNGALGVDIHEKDHPTILEMAKAAGLATGNVSTAELQDATPAALVAHVTSRKCYGPSATSEKCPGNALEKGGKGSITEQLLNARADVTLGGGAKTFAETATAGEWQGKTLREQAQARGYQLVSDAASLNSVTEANQQKPLLGLFADGNMPVRWLGPKATYHGNIDKPAVTCTPNPQRNDSVPTLAQMTDKAIELLSKNEKGFFLQVEGASIDKQDHAANPCGQIGETVDLDEAVQRALEFAKKEGNTLVIVTADHAHASQIVAPDTKAPGLTQALNTKDGAVMVMSYGNSEEDSQEHTGSQLRIAAYGPHAANVVGLTDQTDLFYTMKAALGLKGAPDYKDDDDKGAPHHHHHH

SEQ ID NO: 18 - protein, 215 aa, Artificial Sequence
  Amino acid sequence of light chain Artificial Human Antibody comprising Light Chain Variable Region and Light Chain Constant Region
DIALTQPASVSGSPGQSITISCTGTSSDVGRYNSVSWYQQHPGKAPKLMIYRVSKRPSGVSNRFSGSKSGNTASLTISGLQAEDEADYYCQSWASLSNVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTEA

SEQ ID NO: 19 - DNA, 2073 bp, Artificial Sequence
  DNA sequences of Fd chain with tags
caggtgcaattggtgcagagcggtgccgaagtgaaaaaaccgggcagcagcgtgaaagttagctgcaaagcatccggagggacgttttctggttactacatctcttgggtgcgccaggccccgggccagggcctcgagtggatgggcggtatcatcccgatctctggccgtgcgaactacgcccagaaatttcagggccgggtgaccattaccgccgatgaaagcaccagcaccgcctatatggaactgagcagcctgcgcagcgaagatacggccgtgtattattgcgcgcgttctcgttcttactaccatttcgatctgtggggccaaggcaccctggtgactgttagctcagcgtcgaccaaaggcccgagcgtgtttccgctggccccgagcagcaaaagcaccagcggcggcaccgccgcactgggctgcctggtgaaagattatttcccggaaccagtgaccgtgagctggaacagcggtgccctgaccagcggcgtgcatacctttccggcggtgctgcaaagcagcggcctgtatagcctgagcagcgttgtgaccgtgccgagcagcagcctgggcacccagacctatatttgcaacgtcaaccataaaccgagcaacaccaaagtcgataaaaaagtcgaaccgaaaagcgaattcaaggctgaaatgcctgttctggaaaaccgggctgctcagggcgatattactacacccggcggtgctcgccgtttaacgggtgatcagactgccgctctgcgtgattctcttagcgataaacctgcaaaaaatattattttgctgattggcgatgggatgggggactcggaaattactgccgcacgtaattatgccgaaggtgcgggcggcttttttaaaggtatagatgccttaccgcttaccgggcaatacactcactatgcgctgaatagaaaaaccggcaaaccggactacgtcaccagctcggctgcatcagcaaccgcctggtcaaccggtgtcaaaacctataacggcgcgctgggcgtcgatattcacgaaaaagatcacccaacgattctggaaatggcaaaagccgcaggtctggcgaccggtaacgtttctaccgcagagttgcaggatgccacgcccgctgcgctggtggcacatgtgacctcgcgcaaatgctacggtccgagcgcgaccagtgaaaaatgtccgggtaacgctctggaaaaaggcggaaaaggatcgattaccgaacagctgcttaacgctcgtgccgacgttacgcttggcggcggcgcaaaaacctttgctgaaacggcaaccgctggtgaatggcagggaaaaacgctgcgtgaacaggcacaggcgcgtggttatcagttggtgagcgatgctgcctcactgaactcggtgacggaagcgaatcagcaaaaacccctgcttggcctgtttgctgacggcaatatgccagtgcgctggctaggaccgaaagcaacgtaccatggcaatatcgataagcccgcagtcacctgtacgccaaatccgcaacgtaatgacagtgtaccaaccctggcgcagatgaccgacaaagccattgaattgttgagtaaaaatgagaaaggctttttcctgcaagttgaaggtgcgtcaatcgataaacaggatcatgctgcgaatccttgtgggcaaattggcgagacggtcgatctcgatgaagccgtacaacgggcgctggagttcgctaaaaaggagggtaacacgctggtcatagtcaccgctgatcacgcccacgccagccagattgttgcgccggataccaaagctccgggcctcacccaggcgctaaataccaaagatggcgcagtgatggtgatgagttacgggaactccgaagaggattcacaagaacataccggcagtcagttgcgtattgcggcgtatggcccgcatgccgccaatgttgttggactgaccgaccagaccgatctcttctacaccatgaaagccgctctggggctgaaaggcgcgccggactataaagatgacgatgacaaaggcgcgccgcaccatcatcaccatcac

SEQ ID NO: 20 - DNA, 645 bp, Artificial Sequence
  DNA sequence of Light chain Artificial Human Antibody comprising Light Chain Variable Region and Light Chain Constant Region
gatatcgcgctgacccagccggcgagcgtgagcggtagcccgggccagagcattaccattagctgcaccggcaccagcagcgatgtgggccgttacaactctgtgtcttggtaccagcagcatccgggcaaggcgccgaaactgatgatctaccgtgtttctaaacgtccgagcggcgtgagcaaccgttttagcggatccaaaagcggcaacaccgcgagcctgaccattagcggcctgcaagcggaagacgaagcggattattactgccagtcttgggcttctctgtctaacgttgtgtttggcggcggcacgaagttaaccgttcttggccagccgaaagccgccccaagcgtgaccctgtttccgccgagcagcgaagaactgcaagccaacaaagccaccctggtttgcctgatcagcgatttttatccgggtgccgtgaccgtggcctggaaagccgatagcagcccggtgaaagccggcgtggaaaccaccaccccgagcaaacagagcaacaacaaatatgccgccagcagctatctgagcctgaccccggaacagtggaaaagccatcgcagctatagttgtcaagtgacccatgaaggcagcaccgtggaaaaaaccgtggccccgaccgaggcc

SEQ ID NO: 21 - protein, 691 aa, Artificial Sequence
  Fd chain and tags Artificial Human Antibody comprising Heavy Chain Variable Region; part of Heavy Chain Constant Region; Dimerization Domain Sequence (AP) Flag and HiS6 tags
EVQLVQSGAEVKKPGESLKISCKGSGYSFTGYVIHWVRQMPGKGLEWMGRIDPSKSYTRYSPSFQGQVTISADKSISTAYLQWSSLKASDTAMYYCARGLYSGYFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSEFKAEMPVLENRAAQGDITTPGGARRLTGDQTAALRDSLSDKPAKNIILLIGDGMGDSEITAARNYAEGAGGFFKGIDALPLTGQYTHYALNRKTGKPDYVTSSAASATAWSTGVKTYNGALGVDIHEKDHPTILEMAKAAGLATGNVSTAELQDATPAALVAHVTSRKCYGPSATSEKCPGNALEKGGKGSITEQLLNARADVTLGGGAKTFAETATAGEWQGKTLREQAQARGYQLVSDAASLNSVTEANQQKPLLGLFADGNMPVRWLGPKATYHGNIDKPAVTCTPNPQRNDSVPTLAQMTDKAIELLSKNEKGFFLQVEGASIDKQDHAANPCGQIGETVDLDEAVQRALEFAKKEGNTLVIVTADHAHASQIVAPDTKAPGLTQALNTKDGAVMVMSYGNSEEDSQEHTGSQLRIAAYGPHAANVVGLTDQTDLFYTMKAALGLKGAPDYKDDDDKGAPHHHHHH

SEQ ID NO: 22 - protein, 213 aa, Artificial Sequence
  Amino acid sequence of light chain Artificial Human Antibody comprising Light Chain Variable Region and Light Chain Constant Region
DIELTQPPSVSVSPGQTASITCSGDALGSKYVHWYQQKPGQAPVLVIYAKNNRPSGIPERFSGSNSGNTATLTISGTQAEDEADYYCQSRASGIFGRVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTEA

SEQ ID NO: 23 - DNA, 2073 bp, Artificial Sequence
  DNA sequences of Fd chain with tags
gaagtgcaattggtgcagagcggtgcggaagtgaaaaaaccgggcgaaagcctgaaaattagctgcaaaggctccggatatagcttcactggttacgttatccattgggtgcgccagatgccgggcaaaggtctcgagtggatgggccgtatcgacccgtctaaaagctacacccgttatagcccgagctttcagggccaggtgaccattagcgcggataaaagcatcagcaccgcgtatctgcaatggagcagcctgaaagcgagcgataccgcgatgtattattgcgcgcgtggtctgtactctggttacttcgatatctggggccaaggcaccctggtgactgttagctcagcgtcgaccaaaggcccgagcgtgtttccgctggccccgagcagcaaaagcaccagcggcggcaccgccgcactgggctgcctggtgaaagattatttcccggaaccagtgaccgtgagctggaacagcggtgccctgaccagcggcgtgcatacctttccggcggtgctgcaaagcagcggcctgtatagcctgagcagcgttgtgaccgtgccgagcagcagcctgggcacccagacctatatttgcaacgtcaaccataaaccgagcaacaccaaagtcgataaaaaagtcgaaccgaaaagcgaattcaaggctgaaatgcctgttctggaaaaccgggctgctcagggcgatattactacacccggcggtgctcgccgtttaacgggtgatcagactgccgctctgcgtgattctcttagcgataaacctgcaaaaaatattattttgctgattggcgatgggatgggggactcggaaattactgccgcacgtaattatgccgaaggtgcgggcggcttttttaaaggtatagatgccttaccgcttaccgggcaatacactcactatgcgctgaatagaaaaaccggcaaaccggactacgtcaccagctcggctgcatcagcaaccgcctggtcaaccggtgtcaaaacctataacggcgcgctgggcgtcgatattcacgaaaaagatcacccaacgattctggaaatggcaaaagccgcaggtctggcgaccggtaacgtttctaccgcagagttgcaggatgccacgcccgctgcgctggtggcacatgtgacctcgcgcaaatgctacggtccgagcgcgaccagtgaaaaatgtccgggtaacgctctggaaaaaggcggaaaaggatcgattaccgaacagctgcttaacgctcgtgccgacgttacgcttggcggcggcgcaaaaacctttgctgaaacggcaaccgctggtgaatggcagggaaaaacgctgcgtgaacaggcacaggcgcgtggttatcagttggtgagcgatgctgcctcactgaactcggtgacggaagcgaatcagcaaaaacccctgcttggcctgtttgctgacggcaatatgccagtgcgctggctaggaccgaaagcaacgtaccatggcaatatcgataagcccgcagtcacctgtacgccaaatccgcaacgtaatgacagtgtaccaaccctggcgcagatgaccgacaaagccattgaattgttgagtaaaaatgagaaaggctttttcctgcaagttgaaggtgcgtcaatcgataaacaggatcatgctgcgaatccttgtgggcaaattggcgagacggtcgatctcgatgaagccgtacaacgggcgctggagttcgctaaaaaggagggtaacacgctggtcatagtcaccgctgatcacgcccacgccagccagattgttgcgccggataccaaagctccgggcctcacccaggcgctaaataccaaagatggcgcagtgatggtgatgagttacgggaactccgaagaggattcacaagaacataccggcagtcagttgcgtattgcggcgtatggcccgcatgccgccaatgttgttggactgaccgaccagaccgatctcttctacaccatgaaagccgctctggggctgaaaggcgcgccggactataaagatgacgatgacaaaggcgcgccgcaccatcatcaccatcac

SEQ ID NO: 24 - DNA, 639 bp, Artificial Sequence
  DNA sequence of Light chain Artificial Human Antibody comprising Light Chain Variable Region and Light Chain Constant Region
gatatcgaactgacccagccgccgagcgtgagcgtgagcccgggccagaccgcgagcattacctgtagcggcgatgctctgggttctaaatacgttcattggtaccagcagaaaccgggccaggcgccggtgctggtgatctacgctaaaaacaaccgtccgagcggcatcccggaacgttttagcggatccaacagcggcaacaccgcgaccctgaccattagcggcacccaggcggaagacgaagcggattattactgccagtctcgtgcttctggtatcttcggtcgtgtgtttggcggcggcacgaagttaaccgttcttggccagccgaaagccgccccaagcgtgaccctgtttccgccgagcagcgaagaactgcaagccaacaaagccaccctggtttgcctgatcagcgatttttatccgggtgccgtgaccgtggcctggaaagccgatagcagcccggtgaaagccggcgtggaaaccaccaccccgagcaaacagagcaacaacaaatatgccgccagcagctatctgagcctgaccccggaacagtggaaaagccatcgcagctatagttgtcaagtgacccatgaaggcagcaccgtggaaaaaaccgtggccccgaccgaggcc

(19) United States
(12) Patent Application Publication
(10) Pub. No.: US 2019/0391146 A1
(43) Pub. Date: Dec. 26, 2019

(54) Methods of Detecting and Diagnosing Tuberculosis Using Anti-MTLDL Antibodies

(71) Applicant: The Research Foundation for The State University of New York, Albany, NY (US)

(72) Inventors: Nicole Sampson, Setauket, NY (US); Xinxin Yang, Coram, NY (US)

(21) Appl. No.: 16/481,025

(22) PCT Filed: Jan. 25, 2018
(86) PCT No.: PCT/US2018/015337

§ 371 Date: Jul. 25, 2019

Related U.S. Application Data
(60) Provisional application No. 62/450,845, filed on Jan. 26, 2017.

(51) Int. Cl. G01N 33/569; C07K 16/18; C07K 16/44; G01N 33/92

(52) U.S. Cl. CPC G01N 33/5695; C07K 16/18; C07K 16/44; G01N 2469/10; C07K 2317/21; C07K 2317/55; G01N 33/92

(57) ABSTRACT

The present invention discloses a method for detecting level of Mycobacteria modified Low Density Lipoprotein (MtLDL) in a sample using antibodies or antibody fragments that bind specifically to MtLDL, compositions of such antibodies or antibody fragments and methods of using the same for detecting M. tuberculosis infection in a subject.

### RELATED APPLICATIONS

This application claims the benefit of U.S. Provisional Application No. 62/450,845, filed on Jan. 26, 2017. The entire teachings of the above application are incorporated herein by reference.

### GOVERNMENT SUPPORT

This invention was made with government support under U01HL127522 awarded by the National Institutes of Health. The government has certain rights in the invention.

### BACKGROUND

Mycobacterium tuberculosis (Mtb), the causative agent of tuberculosis (TB), is estimated to infect 30% of the world's population (WHO “Global Tuberculosis Report,” 2016. http://www.who.int/tb/publications/global_report/en/ Last accessed: 20 May 2017.) There are >10 million new cases every year, 11% of which are children less than 15 years old. More than 130,000 children die from TB each year (“Tb in Children,” 2012. http://www.msfaccess.org/content/tb-children Last accessed: 20 May 2017.) The estimated 3 billion people with asymptomatic (latent) TB infections become one of the major contributing factors for the high TB incidence. Clinicians and TB control programs urgently require rapid and more accurate ways to diagnose TB, particularly in children.

### SUMMARY OF INVENTION

In some embodiments, this invention discloses a non-sputum-based enzyme-linked immunosorbent assay (ELISA) assay for detection of M. tuberculosis (Mtb)-modified LDL (MtLDL) as a diagnostic tool for, e.g., active TB disease and latent TB infection. In some embodiments, the assay is a non-sputum based assay.

This invention describes a modified serum lipoprotein (MtLDL) that results from exposure to Mycobacterium tuberculosis (Mtb). MtLDL is different from typical atherosclerotic species like oxidized LDL (Xu, S. et al. Evaluation of Foam Cell Formation in Cultured Macrophages: An Improved Method with Oil Red O Staining and DiI-OxLDL Uptake. Cytotechnology 2010) and acro-LDL (Yoshida, M. et al. Correlation between Images of Silent Brain Infarction, Carotid Atherosclerosis and White Matter Hyperintensity, and Plasma Levels of Acrolein, Il-6 and Crp. Atherosclerosis 2010). Apolipoprotein B (ApoB) from MtLDL is not recognized by anti-acrolein-modified LDL (MDA-LDL) antibodies, suggesting MtLDL contains a modified ApoB that is not an acrolein derivative. It was determined that MtLDL was larger and more positively charged than native LDL, using agarose gel electrophoresis. In contrast, oxidized LDL (OxLDL) and Acrolein modified LDL (MDA-LDL), are more negatively charged than native LDL. (Watanabe, K et al. Acrolein-Conjugated Low-Density Lipoprotein Induces Macrophage Foam Cell Formation. Atherosclerosis 2013) Therefore, the presence of oxidized forms of LDL in patient serum does not interfere with antibody recognition of MtLDL. MtLDL can induce foamy macrophage (FM) formation. Infection of macrophages with non-pathogenic mycobacterial strains, e.g., M. smegmatis, does not result in formation of FMs, whereas infection with M. tuberculosis, M. avium or M. abscessus, or M. bovis can induce FM formation. (Viljoen, A. et al. Mab_3551c Encodes the Primary Triacylglycerol Synthase Involved in Lipid Accumulation in Mycobacterium Abscessus. Mol Microbiol 2016) (Peyron, P., et al. Foamy macrophages from tuberculous patients' granulomas constitute a nutrient-rich reservoir for M. tuberculosis persistence. PLoS Pathog 2008. In one embodiment, the evaluation of FM formation is used as a tool to assess the bioactive components that trigger a tissue response similar to granuloma formation in TB. The results disclosed herein demonstrate that LDL is altered in the presence of Mtb. This modification increases the flux of lipid into activated macrophages, thereby stimulating their conversion into foamy multi-nucleated macrophages that accumulate lipid bodies but have a distinct phenotype from oxLDL or MDA-LDL. Thus, an antibody specific for recognition of MtLDL can be a TB disease-specific diagnostic reagent.

In one embodiment, the invention relates to a method of detecting infection with Mycobacterium tuberculosis (M. tuberculosis or Mtb) in a subject comprising: collecting a test sample from the subject; and detecting Mycobacteria modified Low Density Lipoprotein (MtLDL) in said test sample, using at least one detection agent specific for MtLDL, wherein MtLDL in said test sample is indicative of infection with Mtb. In some other embodiments, the subject is suspected to be infected with Mtb. In a particular embodiment, the subject is suspected to be infected with either latent or active Mtb.

In some embodiments, the invention provides antibodies or antibody fragments, e.g., bivalent Fab fragments, against a Mycobacteria modified serum lipoprotein (MtLDL) that results from exposure to Mycobacterium tuberculosis as a tool for diagnosis of tuberculosis. As used herein, the term “Mycobacteria modified serum lipoprotein” or “mmLP” refers to lipoprotein that is modified specifically in the presence of a Mycobacterial infection and can be used as a biomarker for tuberculosis (TB). In some embodiments of the invention, the methods as described herein comprise detecting levels of a Mycobacteria modified low density lipoprotein (MtLDL). In other embodiments, the methods described herein comprise detecting other species of Mycobacteria modified serum lipoproteins e.g., very low density lipoprotein (VLDL). In one embodiment, the invention provides a method of detecting infection with Mycobacterium tuberculosis (Mtb) in a subject comprising, collecting a test sample from the subject and detecting Mycobacteria modified serum lipoprotein (mmLP) in said test sample, using methods and compositions described herein, wherein the mmLP in said test sample is indicative of infection with Mtb. In another embodiment, the Mycobacteria modified serum lipoprotein (mmLP) is a very low density lipoprotein (VLDL).

In some embodiments, the methods can be used to detect Mycobacteria other than Mycobacterium tuberculosis, e.g., Mycobacteria that can induce FM formation (e.g., M. avium and M. abscessus and M. bovis). For example, in some embodiments, the invention relates to a method of detecting infection with Mycobacterium avium (M. avium) in a subject comprising: collecting a test sample from the subject; and detecting Mycobacteria modified serum Lipoprotein (mmLP) in said test sample, using at least one detection agent specific for mmLP, wherein mmLP in said test sample is indicative of infection with M. avium. In some embodiments, the invention relates to a method of detecting infection with Mycobacterium abscessus (M. abscessus) in a subject comprising: collecting a test sample from the subject; and detecting Mycobacteria modified serum Lipoprotein (mmLP) in said test sample, using at least one detection agent specific for mmLP, wherein mmLP in said test sample is indicative of infection with M. abscessus. In some embodiments, the invention relates to a method of detecting infection with Mycobacterium bovis (M. bovis) in a subject comprising: collecting a test sample from the subject; and detecting Mycobacteria modified Low Density Lipoprotein (MtLDL) in said test sample, using at least one detection agent specific for MtLDL, wherein MtLDL in said test sample is indicative of infection with M. bovis.

The method described herein, can be used to detect mycobacteria modified serum lipoproteins (mmLPs) e.g. Mycobacteria modified LDL or VLDL.

In one embodiment, the method encompasses detecting infection with Mycobacterium tuberculosis (M. tuberculosis) in a subject comprising: collecting a test sample from the subject; and detecting Mycobacteria modified serum lipoprotein (mmLP) in said test sample, using at least one detection agent specific for MtLDL, wherein mmLP in said test sample is indicative of infection with M. tuberculosis.

In one embodiment, the method encompasses detecting infection with Mycobacterium tuberculosis (M. tuberculosis) in a subject comprising: collecting a test sample from the subject; and detecting Mycobacteria modified serum lipoprotein (mmLP) in said test sample, using at least one detection agent for binding mmLP, wherein mmLP in said test sample is indicative of infection with M. tuberculosis.

In one embodiment, the method encompasses detecting infection with Mycobacterium tuberculosis (M. tuberculosis) in a subject comprising: collecting a test sample from the subject; and detecting very low density lipoprotein (VLDL) in said test sample, using at least one detection agent specific for MtLDL, wherein VLDL in said test sample is indicative of infection with M. tuberculosis.

In one embodiment, the method encompasses detecting infection with Mycobacterium tuberculosis (M. tuberculosis) in a subject comprising: collecting a test sample from the subject; and detecting Mycobacteria modified Low Density Lipoprotein (MtLDL) in said test sample, using at least one detection agent specific for MtLDL, wherein MtLDL in said test sample is indicative of infection with M. tuberculosis.

In some embodiments of the methods described herein, the subject is suspected to be infected with latent M. tuberculosis. In some embodiments, the subject is suspected to be infected with active M. tuberculosis. In some embodiments, the subject is 10 years old or under, e.g., the subject is 2 years old or under. In some embodiments, the subject is infected with M. tuberculosis and at least one additional pathogen. In some embodiments, the subject is infected with M. tuberculosis and Human Immune Deficiency virus (HIV). In some embodiments, the subject has an active HIV infection.

In some embodiments, an amount of MtLDL in a test sample of 4 ng/ml or more is indicative of infection with M. tuberculosis.

In some embodiments, the test sample is or comprises blood, sputum, stool, urine, or cerebrospinal fluid (CSF). In one embodiment, the blood is whole blood, serum or plasma. In one embodiment, the test sample comprises serum. In one embodiment, the test sample comprises whole blood. In one embodiment, the test sample comprises plasma. In one embodiment, the test sample comprises stool.

In some embodiments, the inventions also includes kits suitable for detection of infection with M. tuberculosis (Mtb), e.g., in a sample. In some embodiments, the kit comprises at least one binding agent for binding specifically to and form a complex with Mycobacteria modified Low Density Lipoprotein (MtLDL) in the same; a detectable signal-generating compound attached to the binding agent; at least one separation reagent for separating the complex from free, unbound MtLDL and binding agents the sample; and a solid platform. In one embodiment, the kit comprising MtLDL specific antibodies or antibody bivalent Fab fragments thereof. In another embodiment, the kit comprises pairwise combination of MtLDL specific antibodies or antibody bivalent Fab fragments thereof. In another embodiment, the kit comprising binding agents, described herein, can be used for detecting Mycobacteria modified serum lipoprotein (mmLP) in a sample. In some embodiments, the binding agents, described herein, can be used for detecting mmLP in a sample e.g very low density lipoprotein (VLDL).

Disclosed herein is a method of treating infection with M. tuberculosis (Mtb) in a subject is provided. In one embodiment, the method comprises collecting a test sample from the subject and detecting a level of Mycobacteria modified Low Density Lipoprotein (MtLDL) in said test sample using a detection agent specific for MtLDL. In another embodiment, the method comprises diagnosing infection with Mtb, wherein a level of MtLDL in said test sample higher than a corresponding control sample is indicative of infection with Mtb. In a further embodiment, the method comprises administering an appropriate treatment regimen to the subject upon diagnosis of infection with Mtb. In another embodiment, the method of treating infection with Mtb, as described herein, comprises collecting a test sample from the subject and detecting a level of Mycobacteria modified serum lipoprotein (mmLP) in said test sample using a detection agent specific for MtLDL. In another embodiment, the method comprises diagnosing infection with Mtb, wherein a level of mmLP in said test sample higher than a corresponding control sample is indicative of infection with Mtb. In a further embodiment, the method comprises administering an appropriate treatment regimen to the subject upon diagnosis of infection with Mtb. In another embodiment, the mmLP is a very low density lipoprotein (VLDL).

While the invention has been shown and described with reference to certain embodiments of the present invention thereof, it will be understood by those skilled in the art that various changes in from and details may be made therein without departing from the spirit and scope of the present invention and equivalents thereof.

In some embodiments, the methods, as described herein, are used for detecting a mycobacteria modified serum lipoprotein (mmLP) e.g., very low density lipoprotein (VLDL).

Also disclosed herein are methods of using antibodies against a modified serum lipoprotein (MtLDL) that results from exposure to Mycobacterium tuberculosis as a biomarker for diagnosis of tuberculosis. In one example, anti-MtLDL monoclonal antibodies have been generated and a sandwich ELISA was developed using two anti-MtLDL antibodies for detecting MtLDL, e.g., in a sample. In one embodiment, the linear detection range is 15-0.2 ng in serum. In some embodiments, the methods disclosed herein have a hit rate of greater than 30% on TB-positive pediatric serum samples.

In one embodiment, the invention includes a method of selecting a treatment regimen for a subject suspected to be infected with M. tuberculosis and in need thereof, comprising, detecting in a test sample from said subject a level of Mycobacteria modified serum lipoprotein (mmLP); comparing the level of mmLP in the test sample to the corresponding level of mmLP in a control sample; and selecting an appropriate regimen for treating tuberculosis when the level of mmLP in the test sample is greater than the corresponding level of mmLP in control sample. In one embodiment, the mmLP is a very low density lipoprotein (VLDL). In one embodiment, the methods described herein, can be used to detect the level of mycobacteria modified serum lipoprotein (mmLP) other than MtLDL, e.g., very low density lipoprotein (VLDL) in a test sample from a subject.

In one embodiment, the invention relates to a method of determining efficacy of a therapeutic drug for treatment of tuberculosis in a subject in need thereof, comprising, detecting Mycobacteria modified serum lipoproteins (mmLP) in test samples collected from the subject before and after administration of the drug; and comparing the level of mmLP in the test sample collected before administration of the drug with the level of mmLP in the test sample collected after administration of the drug;

wherein a significant reduction in the level of mmLP in the test sample collected after administration of drug compared to the level of mmLP in the test sample collected before administration of drug is indicative of therapeutic efficacy. In yet another embodiment, the methods, as described herein, can be used for detecting a mycobacteria modified serum lipoprotein (mmLP) e.g. very low density lipoprotein (VLDL), in a test sample from a subject.

In some embodiments, the inventions also includes kits suitable for detection of infection with M. tuberculosis (Mtb), e.g., in a sample. In some embodiments, the kit comprises at least one binding agent for binding specifically to and form a complex with Mycobacteria modified Low Density Lipoprotein (MtLDL) in the same; a detectable signal-generating compound attached to the binding agent; at least one separation reagent for separating the complex from free, unbound MtLDL and binding agents the sample; and a solid platform. In one embodiment, the kit comprising MtLDL specific antibodies or antibody bivalent Fab fragments thereof. In another embodiment, the kit comprises pairwise combination of MtLDL specific antibodies or antibody bivalent Fab fragments thereof. In one embodiment, the kit comprising binding agents, described herein, can be used for detecting Mycobacteria modified serum lipoprotein (mmLP) in a sample. In some embodiments, the binding agents, described herein, can be used for detecting mmLP in a sample, e.g., very low density lipoprotein (VLDL).

In another embodiment, the method of treating infection with Mtb, as described herein, comprises collecting a test sample from the subject and detecting a level of Mycobacteria modified serum lipoprotein (mmLP) in said test sample using a detection agent specific for MtLDL. In another embodiment, the method comprises diagnosing infection with Mtb, wherein a level of mmLP in said test sample higher than a corresponding control sample is indicative of infection with Mtb. In a further embodiment, the method comprises administering an appropriate treatment regimen to the subject upon diagnosis of infection with Mtb. In one embodiment, the mmLP is a very low density lipoprotein (VLDL).

### BRIEF DESCRIPTION OF THE DRAWINGS

The patent or application file contains at least one drawing executed in color. Copies of this patent or patent application publication with color drawings will be provided by the Office upon request and payment of the necessary fee.

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1, comprising FIG. 1A and FIG. 1B, illustrates in (A) an agarose gel electrophoresis showing that Mtb modified LDL (MtLDL1 and MtLDL2 were modified by Mtb strains H₃₇Rv and CDC1551, respectively) is larger in size and more positively charged than native LDL; and (B) Immunoblots: blot A showing that apolipoprotein B (ApoB) of MtLDL is larger in size than ApoB of other LDL species (native LDL, oxLDL and acrolein modified LDL, MDA LDL); and blot B showing lack of as cross-reactivity of anti-MDA antibody to MtLDL, thus demonstrating that the modification of MtLDL is different from acrolein-modified LDL.

FIG. 2, comprising FIG. 2A, FIG. 2B and FIG. 2C, illustrates in (FIG. 2A) microscopic images of Oil Red O-hematoxylin stained THP-1 macrophage culture treated with different species of LDL, wherein Mycobacteria modified Low Density Lipoprotein (MtLDL) induces higher lipid body and foamy macrophages (FM) formation in comparison to either Low Density Lipoprotein (LDL), Oxidized LDL (OxLDL) or acrolein modified LDL (MDA-LDL); (FIG. 2B) graph depicting higher level of formation of multi-nucleated THP-1 macrophages by MtLDL in comparison to LDL; and (FIG. 2C) graph depicting formation of a higher concentration of lipid bodies in THP-1 macrophages by MtLDL in comparison to LDL.

FIG. 3 illustrates a method using a direct enzyme-linked immunosorbent assay (ELISA) for determining specificity of 13 selected Mycobacteria modified Low density Lipoprotein (MtLDL) binding bivalent Fab fragments, that were identified from a HuCAL phage display library screen. Eight (8) bivalent Fab fragments displayed high signal/background ratio as depicted on the y-axis as compared to native low density lipoprotein (native LDL), human serum albumin (HSA), glutathione-s-transferase (GST), 6× Histidine (His6-tag) and bovine serum albumin (BSA).

FIG. 4 illustrates a graph showing calibration of a sandwich enzyme-linked immunosorbent assay (ELISA), showing a linear correlation between antigen-specific binding of a pairwise combination of bivalent Fab fragments No. 6 (HCVR SEQ ID NO: 3 and LCVR SEQ ID NO: 4) and Fab fragments No. 8 (HCVR SEQ ID NO: 5 and LCVR SEQ ID NO: 6) (y-axis) to increasing concentration of MtLDL in a MtLDL-spiked cord blood serum sample (x-axis). A lower detection limit of 4-5 ng/mL in serum is shown.

FIG. 5, comprising FIG. 5A, FIG. 5B and FIG. 5C, illustrates in (FIG. 5A) an example schematic of an indirect/sandwich Enzyme Linked Immunosorbent Assay (ELISA) to detect Mycobacteria modified low density lipoprotein (MtLDL) levels in 100 μL infant serum samples for diagnosis of Mycobacterium tuberculosis (Mtb) infection, using a sandwich pair of Fab fragments 6 (HCVR SEQ ID NO: 3 and LCVR SEQ ID NO: 4) and 8 (HCVR SEQ ID NO: 5 and LCVR SEQ ID NO: 6); (FIG. 5B) a curve plot; and (FIG. 5C) a graph showing increase in levels of MtLDL detected in serum from patients clinically diagnosed as likely having latent or active stage tuberculosis disease (TB), as determined by sandwich ELISA using a sandwich pair of Fab fragments 6 (HCVR SEQ ID NO: 3 and LCVR SEQ ID NO: 4) and 8 (HCVR SEQ ID NO: 5 and LCVR SEQ ID NO: 6) the mean serum MtLDL levels of subjects diagnosed with TB disease is 5-fold higher, and subjects diagnosed with Latent TB is 6 to 7-fold higher than serum levels of MtLDL in samples from subjects diagnosed as not having TB (no TB control serum samples).

### DETAILED DESCRIPTION

A description of example embodiments of the invention follows.

Mycobacterium tuberculosis (Mtb), causes >10 million new cases of tuberculosis every year (WHO “Global Tuberculosis Report,”), with a significant proportion of the cases being children (“TB in Children,” 2012). Also, a large proportion of the population with asymptomatic (latent) Mtb infections, become one of the major contributing factors for the high tuberculosis (TB) prevalence. They serve as an immense reservoir of infection, and about 10% of the latent Tuberculosis (latent TB) individual may progress to active TB in their lifetime due to the metabolic state of Mtb (Russell, D. G. et al. Foamy Macrophages and the Progression of the Human Tuberculosis Granuloma. Nat Immunol 2009) (Wakamoto, Y. et al. Dynamic Persistence of Antibiotic-Stressed Mycobacteria. Science 2013) (Murima, P. et al. Targeting Bacterial Central Metabolism for Drug Development. Chem Biol 2014).

There is no gold standard for diagnosing latent TB infection (LTBI) especially in children as current Tuberculosis (TB) diagnostic methods available have been developed for adults, and development efforts have neglected the differences in disease and sampling that occur between adults and children. Clinicians and TB control programs urgently require rapid and more accurate ways to diagnose TB, particularly in children. This need for biomarkers of TB is even more critical in infants and young children, where the diagnostic challenges are greatest and where biological sampling is the most challenging.

The diagnosis rate of childhood TB is very low due to the lack of accurate diagnostic methods for children who cannot produce sputum samples. Current TB diagnostic methods available have been developed for adults, and development efforts have neglected the differences in disease and sampling that occur between adults and children. Young children cannot cough up enough phlegm for a sputum sample. In high-burden settings, most pediatric cases are not bacteriologically confirmed (the established diagnostic reference standard) due to the low diagnostic yield from pediatric samples, the difficulty in obtaining these samples, and limited availability of culture, which has poor sensitivity. There is a large product gap due to the lack of adequately sensitive, organism-based diagnostic tools for TB disease in children, as well as adults.

Current rapid testing methodologies that do not rely on sputum, i.e., tuberculin skin test (TST) and IFN-γ release assays (Quantiferon, IGRA), lack sensitivity in children. (Zar, H. et al. Induced Sputum Versus Gastric Lavage for Microbiological Confirmation of Pulmonary Tuberculosis in Infants and Young Children: A Prospective Study. Lancet 2005) They are the only two currently available methods for diagnosis of LTBI, but they do not distinguish between TB disease and LTBI, and can be affected by confounding factors like recent BCG vaccine administration and non-tuberculous mycobacterial (NTM) infections. (Sharma, S. K. et al. Comparison of Tst and Igra in Diagnosis of Latent Tuberculosis Infection in a High Tb-Burden Setting. PLoS One 2017). If sputum or gastric aspirates are obtained, childhood intrathoracic (pulmonary) TB is usually sputum smear negative; mycobacterial culture (which takes weeks) identifies typically only 30% of pediatric pulmonary TB cases, with only 10-15% being smear-positive (Marais, B. J. et al. Well Defined Symptoms Are of Value in the Diagnosis of Childhood Pulmonary Tuberculosis. Arch Dis Child 2005) (Zar, H. J. et al. Sputum Induction for the Diagnosis of Pulmonary Tuberculosis in Infants and Young Children in an Urban Setting in South Africa. Arch Dis Child 2000). GeneXpert also requires sputum samples, and the sensitivity of the test in smear negative samples is low.

Diagnostic challenges are even greater in Human Immunodeficiency Virus (HIV) co-infected children and infants, (Cotton, M. F. et al. HIV and Childhood Tuberculosis: The Way Forward. Int J Tuberc Lung Dis 2004) where the clinical presentation of pulmonary Tuberculosis (TB) may be non-specific, (Zar, H. J. et al. Induced Sputum Versus Gastric Lavage for Microbiological Confirmation of Pulmonary Tuberculosis in Infants and Young Children: A Prospective Study. Lancet 2005) acute, (Sharma, S. K. et al. Comparison of TST and IGRA in Diagnosis of Latent Tuberculosis Infection in a High Tb-Burden Setting. PLoS One 2017) (Cotton, M. F. et al. Hiv and Childhood Tuberculosis: The Way Forward. Int J Tuberc Lung Dis 2004) and chest radiographs may be atypical. (Marais, B. J. et al. Well Defined Symptoms Are of Value in the Diagnosis of Childhood Pulmonary Tuberculosis. Arch Dis Child 2005) Although rates of bacteriological confirmation appear similar in HIV-infected and uninfected children, (Walters, E. et al. Clinical Presentation and Outcome of Tuberculosis in Human Immunodeficiency Virus Infected Children on Anti-Retroviral Therapy. BMC Pediatr 2008) diagnostic delay and HIV-related immune pathology contribute to higher rates of TB disease progression with increased disease severity, morbidity and mortality. (Jeena, P. M. et al. Effects of the Human Immunodeficiency Virus on Tuberculosis in Children. Int J Tuberc Lung Dis 1996) (Schaaf, H. S. et al. Tuberculosis in Infants Less Than 3 Months of Age. Arch Dis Child 1993) (Jeena, P. M. et al. Impact of Hiv-1 Co-Infection on Presentation and Hospital-Related Mortality in Children with Culture Proven Pulmonary Tuberculosis in Durban, South Africa. Int J Tuberc Lung Dis 2002) (Madhi, S. A. et al. HIV-1 Co-Infection in Children Hospitalised with Tuberculosis in South Africa. Int J Tuberc Lung Dis 2000). Infants, young children, and HIV-infected children are at increased risk of developing TB following infection and of disseminated or severe disease, including TB meningitis. (Marais, B. J. et al. The Natural History of Childhood Intra-Thoracic Tuberculosis: A Critical Review of Literature from the Pre-Chemotherapy Era. Int J Tuberc Lung Dis 2004). The wide spectrum of disease observed in children, and the non-specific signs and symptoms especially in young and HIV+ children, contribute to diagnostic delay and missed opportunities to detect TB, which in turn, favors TB disease progression and poor treatment outcomes. Because TB in children is typically paucibacillary, even if a sputum sample or gastric aspirate is obtained, the utility of GeneXpert is limited.

Antibody-based diagnostics have applications in the field of diagnostics and therapeutics. Just for screening blood donors, there were 16 assays in 70 formats for infectious agents and Human Immunodeficiency Virus (HIV) diagnosis available by 2016. (FDA “Complete List of Donor Screening Assays for Infectious Agents and HIV Diagnostic Assays,” 2016). Antibody diagnostic technology is frequently carried out using enzyme-linked immunosorbent assay (ELISA) technology, which is of low cost and suitable for varied types of end-users including patients and clinicians. One application is the blood glucose meter, which can monitor insulin and glycated hemoglobin (HbA1c) levels using monoclonal antibodies specific to the targets (insulin or HbA1c) in ELISA formats. The method is accurate and suitable even for outpatients. The storage requirements are simple without need for refrigeration and reasonable shelf lives of 18 months. Ultimately, the sandwich ELISA MtLDL-detection technology will be formatted into a test kit for OEM manufacture to distribute and sell as a test for childhood TB disease to help prevent the more than 74,000 pediatric deaths that occur each year from TB. (WHO “Global Tuberculosis Report,” 2016)

Described herein is the identification of Mycobacteria modified LDL (MtLDL) as a biomarker for diagnosing latent tuberculosis in a subject including children. MtLDL is a specific mycobacterial modification of low density lipoprotein (LDL), a host biomolecule. This study shows that MtLDL is different from typical atherosclerotic species like oxidized LDL (OxLDL) (Xu, S. et al. Evaluation of Foam Cell Formation in Cultured Macrophages: An Improved Method with Oil Red O Staining and Dii-OxLDL Uptake. Cytotechnology 2010) and acrolein-modified LDL (MDA-LDL) present in human blood. (Yoshida, M.; Higashi, K.; Kobayashi, E.; Saeki, N.; Wakui, K.; Kusaka, T.; Takizawa, H.; Kashiwado, K.; Suzuki, N.; Fukuda, K. et al. Correlation between Images of Silent Brain Infarction, Carotid Atherosclerosis and White Matter Hyperintensity, and Plasma Levels of Acrolein, IL-6 and CRP. Atherosclerosis 2010) Using agarose gel electrophoresis, the study found that MtLDL is larger/more positively charged than native LDL. In contrast, OxLDL and MDA-LDL are more negatively charged than native LDL. (Watanabe, K.; Nakazato, Y.; Saiki, R.; Igarashi, K.; Kitada, M.; Ishii, I. Acrolein-Conjugated Low-Density Lipoprotein Induces Macrophage Foam Cell Formation. Atherosclerosis 2013) Therefore MtLDL is distinct from known modified LDLs. In addition the study further establishes that apolipoprotein B100 (ApoB100) from MtLDL is not recognized by anti-MDA-LDL antibodies, suggesting MtLDL contains a modified apoB that is not an acrolein derivative, thereby emphasizing the need for development of specific reagents to detect MtLDL to assess whether MtLDL is present in serum from patients with TB disease.

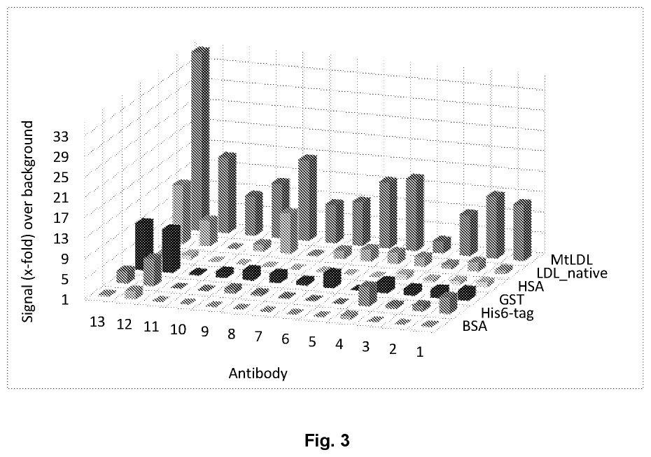

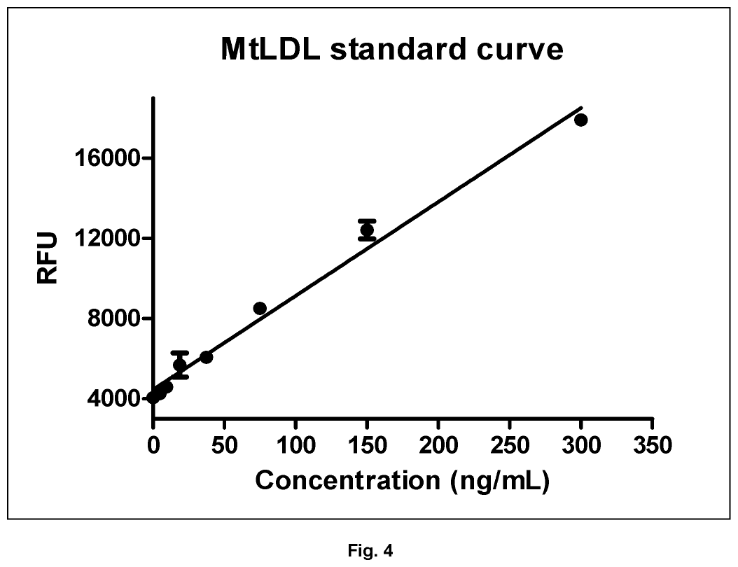

The study herein highlights obtaining 13 anti-MtLDL monoclonal antibodies from the Human Combinatorial Antibody Library (HuCAL) based phage display library, using in vitro selection and counter selection wherein, 8 of the antibodies show a specific signal at least 4-fold above native low density lipoprotein (native LDL) background (FIG. 3). The method disclosed herein, describes screening pairwise combinations of these antibodies to identify antibody pairs that have non-overlapping epitopes suitable for use in a sandwich ELISA. A sandwich ELISA method was calibrated using a pairwise combination of two MtLDL bispecific Fab fragments that shows a linear response of the sandwich ELISA to antigen spiked into pediatric serum, wherein MtLDL is specifically detected at >=4 ppm in the presence of serum LDL, which is around 10⁶ ng/mL (FIG. 4).

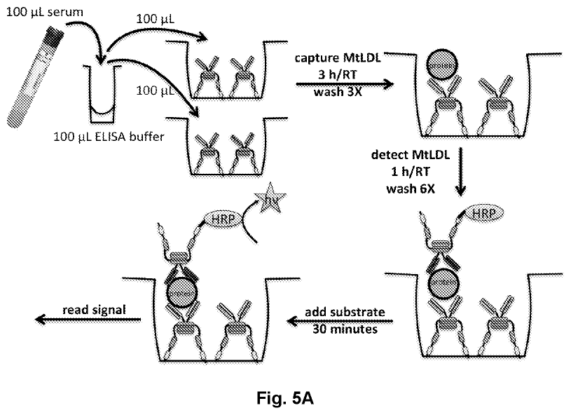

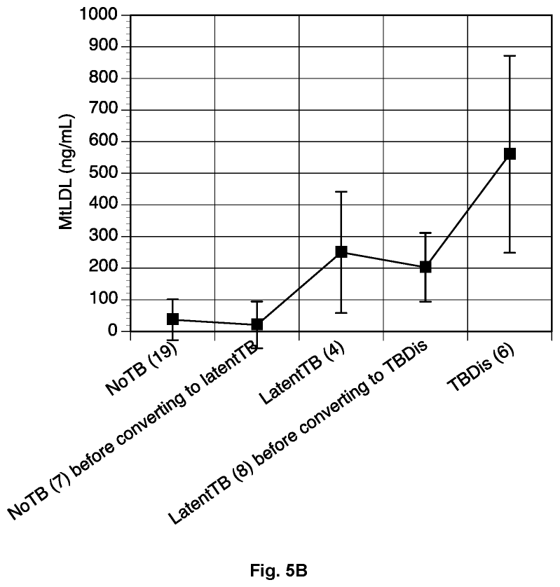

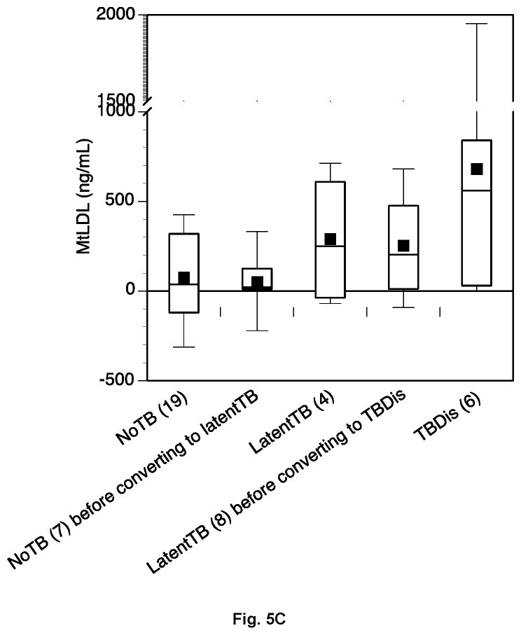

In one embodiment, the methods disclosed herein, describe a rapid and non-sputum-based ELISA detection of Mycobacteria-modified LDL (MtLDL) as a diagnostic tool for active Tuberculosis Disease (TBDis) and latent Tuberculosis (latent TB) infection (FIG. 5A). The method disclosed herein is able to distinguish between TBDis, latent TB and no disease (FIG. 5B-C). MtLDL is a specific mycobacterial modification of low density lipoprotein (LDL), a host biomolecule. This study shows that MtLDL is different from typical atherosclerotic species like oxidized LDL (OxLDL) (Xu, S. et al. Evaluation of Foam Cell Formation in Cultured Macrophages: An Improved Method with Oil Red O Staining and Dii-Oxldl Uptake. Cytotechnology 2010) and acrolein-modified LDL (MDA-LDL) present in human blood (Yoshida, M. et al. Correlation between Images of Silent Brain Infarction, Carotid Atherosclerosis and White Matter Hyperintensity, and Plasma Levels of Acrolein, 11-6 and Crp. Atherosclerosis 2010). Using agarose gel electrophoresis, the study found that MtLDL is larger/more positively charged than native low density lipoprotein (native LDL). In contrast, OxLDL and MDA-LDL are more negatively charged than native LDL (Watanabe, K. et al. Acrolein-Conjugated Low-Density Lipoprotein Induces Macrophage Foam Cell Formation. Atherosclerosis 2013). Therefore MtLDL is distinct from known modified LDLs. In addition the study further establishes that apolipoprotein B100 (ApoB100) from MtLDL is not recognized by anti-MDA-LDL antibodies, suggesting MtLDL contains a modified apoB that is not an acrolein derivative, thereby emphasizing the need for development of specific reagents to detect MtLDL to assess whether MtLDL is present in serum from patients with TB disease.

The antibodies against Mycobacteria modified low density lipoprotein (MtLDL) or anti-MtLDL antibodies, disclosed herein are based on the antigen binding sites of certain antibody F(ab)₂ fragments identified from a Human Combinatorial Antibody based Phage Display screen were selected on the basis of binding to MtLDL (Knappik A. et al. Fully synthetic human combinatorial antibody libraries (HuCAL) based on modular consensus frameworks and CDRs randomized with trinucleotides. J Mol Biol. 2000; Prassler J. et al. HuCAL PLATINUM, a synthetic Fab library optimized for sequence diversity and superior performance in mammalian expression systems. J Mol Biol. 2011). The antibodies contain immunoglobulin variable regions with complementarity-determining region (CDR) sequences that define the antigen binding sites for the anti-MtLDL antibodies MtLDL.

In some embodiments, the antibody F(ab)₂ fragments bind specifically to apolipoprotein B100 (ApoB100) of Mycobacteria modified low density lipoprotein (MtLDL) but does not bind to ApoB of other LDL species. The antibodies disclosed in the study show minimal cross reactivity to native low density lipoprotein (LDL) species associated with disease conditions like atherosclerosis, thereby emphasizing the utility of such antibodies in specifically detecting Mycobacterium tuberculosis (Mtb) infection.

The antibodies against Mycobacteria modified low density lipoprotein (MtLDL) or anti-MtLDL antibodies, disclosed herein are based on the antigen binding sites of certain human antibodies selected on the basis of binding to MtLDL. The antibodies contain immunoglobulin variable regions with complementarity-determining region (CDR) sequences that define the antigen binding sites for the anti-MtLDL antibodies on the MtLDL.

The antibodies described herein show specific binding activity towards Mycobacteria modified low density lipoprotein (MtLDL), and no background non-specific binding activity to non-specific proteins or peptides that could potentially contaminate samples to be tested for the presence of MtLDL e.g., glutathione-S-transferase tag (GST), (6 histidine tag) His6-tag, (influenza virus hemagglutinin tag) HSA, Bovine serum albumin (BSA) and native LDL. In some embodiments the antibodies exhibit significantly higher binding to MtLDL over background non-specific binding to GST, His6-tag, HSA, BSA and native LDL.

Methods of making antibodies and specific antibodies are well-known in the art.

In some embodiments, the antibody is selected from a Human Combinatorial Antibody Library after screening for binding to Mycobacteria modified low density lipoprotein, MtLDL. In some embodiments, the antibody is a completely human antibody, as in neither a humanized nor a chimeric antibody that is substantially free of its natural environment. For instance, antibody or nucleic acid is substantially free of cellular material and other proteins from the cell or tissue source from which it is derived.

As used herein, a Human Combinatorial Antibody Library refers to a HuCAL platinum antibody diversity library containing 4.5×10¹⁰ antibodies. In some embodiments, the antibodies are completely human antibodies. In some embodiments the antibodies comprising the HuCAL library are F(ab)₂ fragments. In some embodiments, the HuCAL diversity library comprises a phage display library, wherein each F(ab)₂ fragment is covalently bound to a phage particle that contains a nucleic acid sequence encoding the same F(ab)₂ fragment that is bound to it. In a further embodiment, upon detecting binding of such phage particle(s) in the library to Mycobacteria modified low density lipoprotein (MtLDL), the nucleic acid is extracted from the phage particle to determine the nucleic and amino acid sequence of the MtLDL binding region of the anti-MtLDL antibody.

In some embodiments, the anti-MtLDL antibody or F(ab)₂ fragment is fused to either 6× Histidine (His6 Tag) or glutathione-s-transferase (GST) Tag or both. In some embodiments, the anti-MtLDL antibody or F(ab)₂ fragment is labelled with an enzyme well known in the art e.g., horse radish peroxidase (HRP), alkaline phosphatase (AP), glucose oxidase, beta-galactosidase (β-Gal) and the like. In another embodiment, the anti-MtLDL antibody or F(ab)₂ fragment is labelled with a fluorochrome, a radioisotope or a chemiluminescent tag.

In some embodiments, the methods of the invention are compatible with protein detection assays that are known to those of skill in the art, such as, for example, immunological and immunochemical methods including, but not limited to, flow cytometry (e.g., FACS analysis), enzyme-linked immunosorbent assays (ELISA), including chemiluminescence assays, radioimmunoassay, immunoblot (e.g., Western blot), immunohistochemistry, immunoprecipitation and other antibody-based quantitative methods. In some embodiments, detection means include chromogenic, chemifluroescent, chemoluminescent and/or radioactive means.

Antibodies that recognize, bind, and/or inhibit MtLDL, including the antibodies and fragments herein, can be used in the methods and kits disclosed herein.

In some embodiments, “antibody” means an intact antibody or antigen-binding fragment of an antibody, e.g., an intact antibody or antigen-binding fragment that is a human antibody. Examples of antibodies that have been modified or engineered are chimeric antibodies, humanized antibodies, multiparatopic antibodies (e.g., biparatopic antibodies), and multi specific antibodies (e.g., bispecific antibodies). Examples of antigen-binding fragments include Fab, Fab′, F(ab′)₂, Fv, single chain antibodies (e.g., scFv), double-stranded Fv (dsFv), minibodies and diabodies.

The antibodies disclosed herein can be whole antibodies or antibody fragments (e.g., a single-chain Fv fragment or Fab antibody fragment), provided that the antibody or antibody fragment is able to recognize and bind to its specific antigen (e.g., MtLDL) in vitro or in vivo. Such antibodies may bind to Mycobacteria modified low density lipoprotein (MtLDL) to detect the presence and level of MtLDL in a sample either naturally containing MtLDL, containing elevated MtLDL or supplemented with MtLDL.

The whole antibodies disclosed herein generally comprise four chains arranged in a classic “Y” motif. There are two heavy chains covalently bound to each other, and two light chains, each covalently bound to one of the heavy chains. The bottom of the “Y” is the Fc region. The two “arms” at the top of the “Y” are the Fab regions. The Fc and the Fab regions are covalently attached. Each Fab region contains a constant region and a variable region (which extends to the tip of the “Y”). Each variable region contains antigen-binding sites (at regions within the variable regions called “hypervariable” regions). Thus, each Fab region has at least one antigen-binding site, and the whole antibody molecule, therefore, has two antigen-binding sites (i.e., is “bivalent”). An antibody fragment comprising two Fab fragments covalently bound is called a F(ab)₂ fragment.

In some embodiments, the antibodies disclosed herein comprise: (a) an immunoglobulin heavy chain variable region comprising the structure CDR_{H1}-CDR_{H2}-CDR_{H3}, and (b) an immunoglobulin light chain variable region comprising the structure CDR_{L1}-CDR_{L2}-CDR_{L3}, wherein the heavy chain variable region and the light chain variable region together define a single binding site for binding Mycobacteria modified low density lipoprotein (MtLDL). Example antibodies of the invention include, but are not limited to anti-MtLDL antibodies described herein (see, e.g., Table 1). Antibodies of the invention include, for example, antibodies having one or more (e.g., 1, 2, 3, 4, 5, or 6) heavy chain complementarity determining regions (CDRs), and/or one or more (e.g., 1, 2, 3, 4, 5, or 6) light chain CDRs of the anti-MtLDL antibodies. Thus, in an embodiment, the invention provides an antibody, or antigen-binding fragment, comprising an antibody heavy chain variable (V_{H}) domain having at least one (e.g., 1, 2, 3, 4, 5, or 6) CDR selected from the group consisting of: a heavy chain variable region (HCVR) comprising complementarity determining regions (CDRs) selected from the group consisting of: CDRs 1-3 of SEQ ID NO: 1, CDRs 1-3 of SEQ ID NO: 3, CDRs 1-3 of SEQ ID NO: 5, and CDRs 1-3 of SEQ ID or NO: 7.

In some embodiments, CDRs 1-3 refer to the three underlined portions of a variable region in Table 1 disclosed herein. The first underlined region is CDR 1, the second is CDR 2 and the third is CDR 3.

In an embodiment, the invention provides an antibody, or antigen-binding fragment, comprising an antibody light chain variable (V_{L}) domain having at least one (e.g., 1, 2, 3, 4, 5, or 6) CDR selected from the group consisting of: a Light chain variable region (LCVR) comprising complementarity determining regions (CDRs) selected from the group consisting of: CDRs 1-3 of SEQ ID NO: 2, CDRs 1-3 of SEQ ID NO: 4, CDRs 1-3 of SEQ ID NO: 6, and CDRs 1-3 of SEQ ID or NO: 8.

In some embodiments, the antibody, or antigen-binding fragment, comprises an antibody variable light chain (V_{L}) domain having at least one complementarity determining region (CDR) of the Mycobacteria modified low density lipoprotein (MtLDL) antibodies. Thus, in an embodiment, the invention provides an antibody, or antigen-binding fragment, comprising an antibody light chain variable (V_{L}) domain having at least one (e.g., 1, 2, 3, 4, 5, or 6) CDR selected from the group consisting of: an antibody heavy chain variable (V_{H}) domain having at least one (e.g., 1, 2, 3, 4, 5, or 6) CDR. In one embodiment, the light chain variable region (LCVR) comprising CDRs selected from the group consisting of: CDRs 1-3 of SEQ ID NO: 2, CDRs 1-3 of SEQ ID NO: 4, CDRs 1-3 of SEQ ID NO: 6, and CDRs 1-3 of SEQ ID NO: 8; In particular embodiments, the antibody, or antigen-binding fragment, has all three heavy chain CDRs of the anti-MtLDL antibody (SEQ ID NO: 1; SEQ ID NO: 3; SEQ ID NO: 5; and SEQ ID NO: 7).

In certain embodiments, an antibody of the invention comprises all three VH CDRs (SEQ ID NO:1; SEQ ID NO:3; SEQ ID NO:5 and SEQ ID NO:7) and all three VL CDRs (SEQ ID NO:2; SEQ ID NO:4; SEQ ID NO:6 and SEQ ID NO:8) of the MtLDL antibody (Table 1).

In one embodiment, invention provides an antibody, or antigen-binding fragment, comprising an antibody heavy chain variable (V_{H}) domain having at least one (e.g., 1, 2, 3, 4, 5, or 6) CDR selected from the group consisting of: a heavy chain variable region (HCVR) comprising complementarity determining regions (CDRs) selected from the group consisting of: CDRs 1-3 of SEQ ID NO: 9, CDRs 1-3 of SEQ ID NO: 13, CDRs 1-3 of SEQ ID NO: 17, and CDRs 1-3 of SEQ ID or NO: 21.

In some embodiment, invention provides an antibody, or antigen-binding fragment, comprising an antibody light chain variable (V_{L}) domain having at least one (e.g., 1, 2, 3, 4, 5, or 6) CDR selected from the group consisting of: a light chain variable region (LCVR) comprising complementarity determining regions (CDRs) selected from the group consisting of: CDRs 1-3 of SEQ ID NO: 10, CDRs 1-3 of SEQ ID NO: 14, CDRs 1-3 of SEQ ID NO: 18, and CDRs 1-3 of SEQ ID or NO: 22.

In some embodiments, the light chain variable region (LCVR) comprising CDRs selected from the group consisting of: CDRs 1-3 of SEQ ID NO: 10, CDRs 1-3 of SEQ ID NO: 14, CDRs 1-3 of SEQ ID NO: 18, and CDRs 1-3 of SEQ ID NO: 22; In particular embodiments, the antibody, or antigen-binding fragment, has all three heavy chain CDRs of the anti-MtLDL antibody (SEQ ID NO: 9; SEQ ID NO: 13; SEQ ID NO: 17; and SEQ ID NO: 21). In certain embodiments, an antibody of the invention comprises all three VH CDRs (SEQ ID NO:9; SEQ ID NO:13; SEQ ID NO:17 and SEQ ID NO:21) and all three VL CDRs (SEQ ID NO:10; SEQ ID NO:14; SEQ ID NO:18 and SEQ ID NO:22) of the MtLDL antibody (Table 1).

In one embodiment, the invention provides anti-MtLDL, bivalent Fab antibody fragments, comprising of two binding sites for MtLDL, wherein each binding site comprises (a) an immunoglobulin Fd chain comprising the structure heavy chain variable region comprising the structure CDR_{H1}-CDR_{H2}-CDR_{H3} followed by a portion of the heavy chain constant region; and (b) an immunoglobulin light chain comprising the structure CDR_{L1}-CDR_{L2}-CDR_{L3} followed by the light chain constant region, wherein the variable region of the Fd chain and the variable region of the light chain together define a single binding site for binding Mycobacteria modified low density lipoprotein (MtLDL). In some embodiments of the invention, the anti-MtLDL, bivalent Fab antibody fragments, comprise (a) an immunoglobulin Fd chain comprising the structure heavy chain variable region comprising all three VH CDRs (SEQ ID NO:9; SEQ ID NO:13; SEQ ID NO:17 and SEQ ID NO:21) and all three VL CDRs (SEQ ID NO:10; SEQ ID NO:14; SEQ ID NO:18 and SEQ ID NO:22) of the MtLDL antibody (Table 1). In some embodiments, the anti-MtLDL, bivalent Fab antibody f, comprise (a) an immunoglobulin Fd chain comprising the heavy chain variable region (SEQ ID NO:9; SEQ ID NO:13; SEQ ID NO:17 and SEQ ID NO:21) and a variable light chain region (SEQ ID NO:10; SEQ ID NO:14; SEQ ID NO:18 and SEQ ID NO:22) of the MtLDL antibody (Table 1).

In one embodiment, the anti-MtLDL, bivalent Fab antibody, binds to MtLDL at an epitope that is recognized by a heavy chain variable regions of the immunoglobulin Fd chains (SEQ ID NO:9; SEQ ID NO:13; SEQ ID NO:17 and SEQ ID NO:21); and a variable light chain region (SEQ ID NO:10; SEQ ID NO:14; SEQ ID NO:18 and SEQ ID NO:22).

A person of ordinary skill in the art would recognize that, in some instances, it may be possible to combine one or more CDRs from the one anti-MtLDL antibody with one or more CDRs from another anti-MtLDL antibody (either on the same chain or different chains) to produce an antibody that retains binding (e.g., specific binding) to MtLDL. Accordingly, the invention encompasses antibodies having CDRs from a combination of more than one anti-MtLDL antibodies, in any combination (e.g., a heavy chain comprising VH CDR1 and 3 from SEQ ID NO: 1 and the VH CDR2 from SEQ ID NO: 3).

In some embodiments, an antibody of the invention is a bispecific antibody wherein each arm of the anti-LDL antibody binds to different epitopes on Mycobacteria modified low density lipoprotein (MtLDL) protein comprising combination of any two LCVR/HCVR for e.g. SEQ ID NO: 1/SEQ ID NO: 2 and SEQ ID NO: 3/SEQ ID NO:4. Additional functional HCVR/LCVR pairings that are within the scope of the invention.

In some embodiments, an antibody of the invention comprises combinations of two heavy chain variable regions (HCVR) chosen from the anti-MtLDL antibody sequences (SEQ ID NO: 1; SEQ ID NO: 3; SEQ ID NO: 5 and SEQ ID NO: 7), combinations of two light chain variable region (LCVR) chosen from the anti-MtLDL antibody sequences (SEQ ID NO: 2; SEQ ID NO: 4; SEQ ID NO: 6 and SEQ ID NO: 8), or both. Thus, possible HCVR/LCVR combinations for an antibody of the invention include SEQ ID NO:1/SEQ ID NO:4; SEQ ID NO:1/SEQ ID NO:6; SEQ ID NO:1/SEQ ID NO:8; SEQ ID NO:3/SEQ ID NO:2; SEQ ID NO:3/SEQ ID NO:6; SEQ ID NO:3/SEQ ID NO:8; SEQ ID NO:5/SEQ ID NO:2; SEQ ID NO:1/SEQ ID NO:4 SEQ ID NO:5/SEQ ID NO:8; and additional functional HCVR/LCVR pairings that are within the scope of the invention.

In some embodiments, the antibody binds specifically to a mammalian Mycobacteria modified low density lipoprotein MtLDL (e.g., human MtLDL, or hMtLDL). This means that the antibody binds to MtLDL protein in a sample, with negligible binding to other proteins present in the sample, under a given set of binding reaction conditions.

In some embodiments, the antibody binds specifically to MtLDL but does not bind to native LDL, oxidized LDL (ox-LDL) and acrolein LDL (MDA-LDL). This means that the antibody binds to MtLDL in a sample, with negligible binding to other LDL species present in the sample, under a given set of binding reaction conditions.

In some embodiments, the antibody preferentially binds MtLDL, e.g., binds with a greater than 5-fold specificity for tdLDL.

Examples of antigen-binding fragments of an antibody include, a Fab, Fab′, F(ab′)₂, Fv, scFv, dsFv, dAb, and a diabody.

A “Fab fragment” comprises one light chain and the CH1 and variable regions of one heavy chain. The heavy chain of a Fab molecule cannot form a disulfide bond with another heavy chain molecule.

An “Fc” region contains two heavy chain fragments comprising the CH2 and CH3 domains of an antibody. The two heavy chain fragments are held together by two or more disulfide bonds and by hydrophobic interactions of the CH3 domains.

A “Fab′ fragment” contains one light chain and a portion of one heavy chain that contains the VH domain and the CH1 domain and also the region between the CH1 and CH2 domains, such that an interchain disulfide bond can be formed between the two heavy chains of two Fab′ fragments to form a F(ab′)₂ molecule.

A “F(ab′)₂ fragment” contains two light chains and two heavy chains containing a portion of the constant region between the C_{H}1 and C_{H}2 domains, such that an interchain disulfide bond is formed between the two heavy chains. A F(ab′)₂ fragment thus is composed of two Fab′ fragments that are held together by a disulfide bond between the two heavy chains.

The “Fv region” comprises the variable regions from both the heavy and light chains, but lacks the constant regions.

A “single-chain Fv antibody” (or “scFv antibody”) refers to antibody fragments comprising the VH and VL domains of an antibody, wherein these domains are present in a single polypeptide chain. Generally, the Fv polypeptide further comprises a polypeptide linker between the VH and VL domains which enables the scFv to form the desired structure for antigen binding.

A dsFv fragment consists of a Fab fragment minus the constant regions, i.e., consisting only of the variable regions of a heavy and light chain covalently bound to each other.

Classically, both dsFv and scFv fragments are monovalent (and thus mono-specific). However, two dsFv fragments or two scFv fragments can themselves be linked to form a bispecific fragment (which would be analogous to an F(ab′)2 fragment without the constant regions). Furthermore, it is possible to link two dsFv fragments or scFv fragments with different antigen-binding sites (i.e., different specificities), to form a bi-specific fragment.

A “diabody” is a small antibody fragment with two antigen-binding sites. The fragments comprise a heavy chain variable region (VH) connected to a light chain variable region (VL) in the same polypeptide chain (VH-VL or VL-VH). By using a linker that is too short to allow pairing between the two domains on the same chain, the domains are forced to pair with the complementary domains of another chain and create two antigen-binding sites.

A “domain antibody fragment” is an immunologically functional immunoglobulin fragment containing only the variable region of a heavy chain or the variable region of a light chain. In some instances, two or more VH regions are covalently joined with a peptide linker to create a bivalent domain antibody fragment. The two VH regions of a bivalent domain antibody fragment may target the same or different antigens.

In some embodiments, the antibody is modified or engineered. Examples of modified or engineered antibodies include chimeric antibodies, multiparatopic antibodies (e.g., biparatopic antibodies), and multispecific antibodies (e.g., bispecific antibodies).

As used herein, “multiparatopic antibody” means an antibody that comprises at least two single domain antibodies, in which at least one single domain antibody is directed against a first antigenic determinant on an antigen and at least one other single domain antibody is directed against a second antigenic determinant on the same antigen. Thus, for example, a “biparatopic” antibody comprises at least one single domain antibody directed against a first antigenic determinant on an antigen and at least one further single domain antibody directed against a second antigenic determinant on the same antigen.

As used herein, “multispecific antibody” means an antibody that comprises at least two single domain antibodies, in which at least one single domain antibody is directed against a first antigen and at least one other single domain antibody is directed against a second antigen (different from the first antigen). Thus, for example, a “bispecific” antibody is one that comprises at least one single domain antibody directed against a first antigen and at least one further single domain antibody directed against a second antigen, e.g., different from the first antigen.

In some embodiments, the antibodies disclosed herein are monoclonal antibodies, e.g., a human or non-human monoclonal antibodies (e.g., rat, rabbit, murine or other non-human animal).

In some embodiments, disclosed herein is a method for detecting the level of Mycobacteria modified low density lipoprotein (MtLDL) in a test sample from a subject. The method disclosed herein, comprises capturing MtLDL in a test sample onto a solid phase by incubating with a first antibody or antibody fragment that binds specifically to an epitope of MtLDL, for a time and condition suitable for binding, wherein the first antibody or antibody fragment is attached to the solid phase support. An example method includes incubating the test sample with the first antibody for 3 hours at room temperature (20-25° C.). The method further comprises separating the MtLDL/first antibody complex from free, unbound antigens in a test sample by washing the solid phase support with a suitable buffer for a suitable number of time, followed by detecting levels of MtLDL in the test sample by incubating with a second antibody of antibody fragment that binds specifically to another epitope on MtLDL, for a time and condition suitable for binding. Example method includes washing the solid phase support with a washing buffer for 3 times at room temperature. In some embodiments, the method comprises detecting the binding of the second antibody or antibody fragment to MtLDL by using a third labelled antibody or antibody fragment or a labelled compound. In some embodiments, the second antibody is labelled. In some embodiments, the label is a detectable tag for detection by another antibody e.g. a 6 Histidine (His6 tag); a glutathione-s-transferase tag (GST), a myc tag, a FLAG tag or an influenza virus hemagglutinin tag (HA tag). In some embodiments, the label is an enzyme that upon reacting with a substrate generates a detectable signal e.g. horse radish peroxidase (HRP), alkaline phosphatase (AP), glucose oxidase, beta-galactosidase (β-Gal). In some embodiments, the label is a fluorochrome, a radioisotope or a chemiluminescent tag.

In some embodiments, the antibody or antibody fragment binds to the same epitope of Mycobacteria modified low density lipoprotein (MtLDL) as an antibody or fragment thereof comprising the heavy chain variable region (HVCR) of SEQ ID NO: 1 and the light chain variable region (LCVR) of SEQ ID NO: 2, the HVCR of SEQ ID NO: 3 and the LCVR of SEQ ID NO: 4, the HVCR of SEQ ID NO: 5 and the LCVR of SEQ ID NO: 6, or the HVCR of SEQ ID NO: 7 and the LCVR of SEQ ID NO: 8. In some embodiments, the antibody or antibody fragment competitively inhibits binding of an antibody or fragment thereof comprising the heavy chain variable region (HVCR) of SEQ ID NO: 1 and the light chain variable region (LCVR) of SEQ ID NO: 2, the HVCR of SEQ ID NO: 3 and the LCVR of SEQ ID NO: 4, the HVCR of SEQ ID NO: 5 and the LCVR of SEQ ID NO: 6, or the HVCR of SEQ ID NO: 7 and the LCVR of SEQ ID NO: 8. In some embodiment, the antibody or antibody fragment comprises the heavy chain variable region (HVCR) of SEQ ID NO: 1 and the light chain variable region (LCVR) of SEQ ID NO: 2, the HVCR of SEQ ID NO: 3 and the LCVR of SEQ ID NO: 4, the HVCR of SEQ ID NO: 5 and the LCVR of SEQ ID NO: 6, or the HVCR of SEQ ID NO: 7 and the LCVR of SEQ ID NO: 8.

In some embodiments, the second antibody or antibody fragment comprises either one or both of a heavy chain variable region (HCVR) comprising complementarity determining regions (CDRs) selected from the group consisting of: CDRs 1-3 of SEQ ID NO: 1, CDRs 1-3 of SEQ ID NO: 3, CDRs 1-3 of SEQ ID NO: 5, and CDRs 1-3 of SEQ ID and NO: 7; and a light chain variable region (LCVR) comprising CDRs selected from the group consisting of: CDRs 1-3 of SEQ ID NO: 2, CDRs 1-3 of SEQ ID NO: 4, CDRs 1-3 of SEQ ID NO: 6, and CDRs 1-3 of SEQ ID NO: 8. In some embodiments, second antibody or antibody fragment comprises either one or both of a heavy chain variable region (HCVR) comprising an amino acid sequence selected from the group consisting of: SEQ ID NO: 1, SEQ ID NO: 3, SEQ ID NO: 5, and SEQ ID NO: 7; and a light chain variable region (LCVR) comprising an amino acid sequence selected from the group consisting of: SEQ ID NO: 2, SEQ ID NO: 4, SEQ ID NO: 6, and SEQ ID NO: 8.

In some embodiments, the method of detecting level of Mycobacteria modified low density lipoprotein (MtLDL) in a test sample from a subject comprises a pairwise combination of two antibodies or fragments thereof as first and second antibodies, wherein each antibody or fragment thereof comprises an amino acid sequence different from the other. In some embodiments, the pairwise combination is: an antibody comprising an HCVR of SEQ ID NO: 1 and an LCVR of SEQ ID NO: 2 paired with an antibody comprising an HCVR of SEQ ID NO: 3 and an LCVR of SEQ ID NO: 4; an antibody comprising an HCVR of SEQ ID NO: 1 and an LCVR of SEQ ID NO: 2 paired with an antibody comprising an HCVR of SEQ ID NO: 5 and an LCVR of SEQ ID NO: 6; an antibody comprising an HCVR of SEQ ID NO: 1 and an LCVR of SEQ ID NO: 2 paired with an antibody comprising an HCVR of SEQ ID NO: 7 and an LCVR of SEQ ID NO: 8; an antibody comprising an HCVR of SEQ ID NO: 3 and an LCVR of SEQ ID NO: 4 paired with an antibody comprising an HCVR of SEQ ID NO: 5 and an LCVR of SEQ ID NO: 6; an antibody comprising an HCVR of SEQ ID NO: 3 and an LCVR of SEQ ID NO: 4 paired with an antibody comprising an HCVR of SEQ ID NO: 7 and an LCVR of SEQ ID NO: 8; or an antibody comprising an HCVR of SEQ ID NO: 5 and an LCVR of SEQ ID NO: 6 paired with an antibody comprising an HCVR of SEQ ID NO: 7 and an LCVR of SEQ ID NO: 8.

In some embodiments, the pairwise combination is an antibody comprising an HCVR of SEQ ID NO: 3 and an LCVR of SEQ ID NO: 4 paired with an antibody comprising an HCVR of SEQ ID NO: 5 and an LCVR of SEQ ID NO: 6. Example pairwise combination of the invention include a first antibody comprising an HCVR of SEQ ID NO: 5 and an LCVR of SEQ ID NO: 6 paired with a second antibody comprising an HCVR of SEQ ID NO: 3 and an LCVR of SEQ ID NO: 4.

In some embodiments, one of the antibodies in the pairwise combination of two antibodies or fragments thereof is affixed to a solid matrix. In some embodiments, the solid matrix is a microtiter plate made of poly-styrene, poly-propylene, poly-vinyl or poly-olefin matrix.

A method of detecting infection with Mycobacterium tuberculosis (Mtb) in a subject comprising: collecting a test sample from the subject; detecting Mycobacteria modified Low Density Lipoprotein (MtLDL) in said test sample, using at least one detection agent specific for MtLDL, wherein MtLDL in said test sample is indicative of infection with Mtb. In one embodiment the subject is infected with latent Mtb. In one embodiment, the subject is infected with active Mtb.

The subject can be an adult, child or infant. In one embodiment, the subject is 12 years old and under. In another embodiment, the subject is 10 years old or under. In yet another, embodiment, the subject is 2 years old and under. One Example embodiment shows detection of an amount of MtLDL in a test sample from a subject of 4 ng/ml or more is indicative of infection with Mtb. In one embodiment, the subject has a positive IGRA result or a TST reaction of 5 or more millimeters.

In some embodiments, the method for detecting Mycobacteria modified low density lipoprotein (MtLDL) in a test sample is used to diagnose infection with M. tuberculosis (Mtb), wherein a level of MtLDL in the test sample higher than a level of MtLDL in a control sample is indicative of infection with Mtb. In some embodiments, the method of detecting MtLDL in a test sample is used to compare the level of MtLDL between a test sample from a subject suspected to me infected with Mtb and a control sample from a subject who is healthy. In some embodiments, the level of MtLDL in a test sample is detected as fold change over the corresponding amount of MtLDL in a control sample. In another embodiment, a 4-fold increase in the amount of MtLDL in a test sample over the corresponding amount of MtLDL in a control sample is indicative of infection with Mtb.

Also disclosed herein, is a method of selecting a treatment regimen for a subject suspected to be infected with Mycobacterium tuberculosis (Mtb) and in need thereof, comprising: detecting in a test sample from said subject a level of Mycobacteria modified low density lipoprotein (MtLDL); comparing the level of MtLDL in the test sample to a corresponding level of MtLDL in a control sample; and selecting an appropriate regimen for treating tuberculosis when level of MtLDL in test sample is greater than the corresponding level of MtLDL in control sample. In one embodiment, a selected appropriate treatment regimen is administered to the subject. In one embodiment, an appropriate treatment regimen for a subject in need thereof comprises a standard treatment regimen as approved and recommended by Center of Disease Control and Prevention (Official American Thoracic Society/Centers for Disease Control and Prevention/Infectious Diseases Society of America Clinical Practice Guidelines: Treatment of Drug-Susceptible Tuberculosis Nahid, P. et al., Infectious Disease Society of America, IDSA Guidelines, Aug. 10, 2016) and WHO guidelines (Treatment of Tuberculosis: Guidelines. 4th edition. Geneva: World Health Organization; 2010).

In one embodiment, standard treatment regimen as approved for treatment for TB Disease by Center of Disease Control and Prevention comprises the following: TB disease can be treated by taking several drugs for 6 to 9 months. There are 10 drugs currently approved by the United States Food and Drug Administration (USFDA) for treating TB. Of the approved drugs, the first-line anti-TB agents that form the core of treatment regimens comprise an Intensive phase of administration of: isoniazid (INH), rifampin (RIF), ethambutol (EMB) and pyrazinamide (PZA) at doses and intervals: e.g., 7 days/week for 14 doses then twice weekly for 12 doses; 3 times weekly for 24 doses (8 weeks); 7 days/week for 56 doses (8 weeks) or 5 days/week for 40 doses (8 weeks); 7 days/week for 56 doses (8 weeks) or 5 days/week for 40 doses (8 weeks). Each of these treatment is followed by Continuation Phase treatment comprising administration of: isoniazid (INH) and rifampin (RIF) at doses and intervals: Twice weekly for 36 doses (18 weeks); 3 times weekly for 54 doses (18 weeks); 3 times weekly for 54 doses (18 weeks); 7 days/week for 126 doses (18 weeks) or 5 days/week for 90 doses (18 weeks), respectively.

A standard treatment regimen as approved for treatment for Latent TB by Center of Disease Control and Prevention comprises the following: Isoniazid, 9 months, daily to 6 months twice weekly; Isoniazid and Rifapentine. 3 months, once weekly; Rifampin. 4 months daily. Treatment of latent TB infection should be initiated after the possibility of TB disease has been excluded.

Also disclosed herein, is a method for determining efficacy of a therapeutic drug for treatment of tuberculosis in a subject in need thereof, comprising: detecting Mycobacteria modified low density lipoprotein (MtLDL) test samples collected from the subject before and after administration of the drug; comparing the level of MtLDL in the test sample collected before administration of the drug with the level of MtLDL in the test sample collected after administration of the drug. In one embodiment, a significant reduction in the level of MtLDL in the test sample collected after administration of drug compared to the level of MtLDL in the test sample collected before administration of drug is indicative of therapeutic efficacy. In some embodiments, the therapeutic drug is an antibiotic, a small molecule drug, a chemical compound, a peptide or fragment thereof, an antibody or fragment thereof, a nucleic acid molecule or a combination thereof.

In one embodiment, the invention includes a kit suitable for detecting a level of MtLDL in a sample. In one embodiment, the kit comprises: at least one binding agent. for binding specifically to and form a complex with MtLDL in the same; a detectable signal-generating compound attached to the binding agent; at least one separation reagent for separating the complex from free, unbound MtLDL and binding agents the sample; and a solid platform. In one embodiment, the kit is used for detecting infection with Mtb in subject. In one embodiment a subject is diagnosed with tuberculosis if the level of MtLDL as measured by the kit, in a sample from the subject suspected to be infected with Mtb is significantly higher than that of a control sample from a healthy subject. In another embodiment, appropriate treatment regimen is administered to the subject upon diagnosis of infection with Mtb.

A method of treating infection with Mycobacterium tuberculosis (Mtb) in a subject comprising: collecting a test sample from the subject; detecting a level of Mycobacteria modified Low Density Lipoprotein (MtLDL) in said test sample using a detection agent specific for MtLDL, diagnosing infection with Mtb, wherein a level of MtLDL in said test sample higher than a corresponding control sample is indicative of infection with (Mtb); and administering an appropriate treatment regimen to the subject upon diagnosis of infection with (Mtb).

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

A description of example embodiments follows. The following Examples are merely illustrative, and are not intended to limit the scope or content of the invention in any way.

Cites

### Material and Reagents

Bacterial Strains and Culture Conditions.

Mycobacterium tuberculosis (M. tb) strain CDC1551 was cultured at 37° C. in Middlebrook 7H9 (broth) supplemented with 0.2% glycerol or 0.1 mM cholesterol, 0.5% BSA, 0.08% NaCl, 0.05% (v/v) tyloxapol.

Preparation of LDL.

HepG2 human liver cells (ATCC HB-8065) were grown to 80% confluence in HepG2 growth media (DMEM, 10% fetal bovine serum, 20 mM L-glutamine, 100 U/mL penicillin, 100 μg/mL streptomycin, and 10 mM HEPES). Cells were grown for 4-5 days. Culture supernatants were then harvested and concentrated. LDL particles were separated by density gradient ultracentrifugation and desalted by ultrafiltration through a 100-kDa molecular weight filter. LDL was sterile filtered before use in experiments.

Mtb-Modified LDL (MtLDL).

Mycobacterium tuberculosis (M. tb) CDC1551 was cultured to OD˜0.7 and LDL was added. The culture was incubated at 37° C. for 7 days. The Mycobacteria-modified low density lipoprotein (MtLDL) was isolated from the culture by sterile filtration of the supernatant, and MtLDL was further concentrated by ultrafiltration through a 100-kDa molecular weight cutoff filter and washed four times with phosphate buffered saline (PBS). MtLDL was sterile filtered before use in experiments.

Generation of Anti-mtLDL Antibodies.

Monoclonal antibodies against Mycobacteria-modified low density lipoprotein (MtLDL) (bivalent Fab) with appropriate detection tags were identified utilizing the Human Combinatorial Antibody Library (HuCAL, Bio-Rad AbD Serotec GmbH). The HuCAL phage display library was depleted of antibodies that recognize intact LDL. The depleted library was panned for three rounds of binding, elution, and amplification to isolate antibodies specific for MtLDL.

Sandwich ELISA Assay.

Briefly, 96 well plates were coated overnight at 4° C. or 1-3 hours at 37° C. with 1-15 μg/mL (e.g. 5 μg/mL) capture antibody (e.g., SEQ ID NO. 6) in phosphate buffered saline (PBS) or any standard coating buffers. Plates were washed with PBS containing 0.05% Tween-20 (PBST) or any standard buffers for 3-5 times, blocked with 3% Bovine serum albumin (BSA) in PBST or any standard blocking buffers for 1-3 hours at room temperature or overnight at 4° C., loaded serial dilutions of standards and test samples in HISPEC assay diluent (Bio-Rad) or any standard diluent buffers, and allowed to react for 1-24 hours at 4-37° C. Plates were then washed 3-5 times and treated with 0.5-10 μg/mL (e.g. 2 μg/mL) of HRP- or AP-conjugated detection antibody in HISPEC assay diluent or any standard diluent buffers for 1-3 hours at room temperature. After washing, the plates were developed with HRP or AP detection reagents, e.g., a QuantaBlu fluorogenic peroxidase substrate kit (Thermo Scientific), for 30 min at room temperature. Fluorescence was recorded (ex. 320±25 nm, em. 430±35 nm).

### Example 1: Characterization of Mycobacterium tuberculosis Modified Low Density Lipoprotein as a Host Bio-Marker for Diagnosis of Tuberculosis

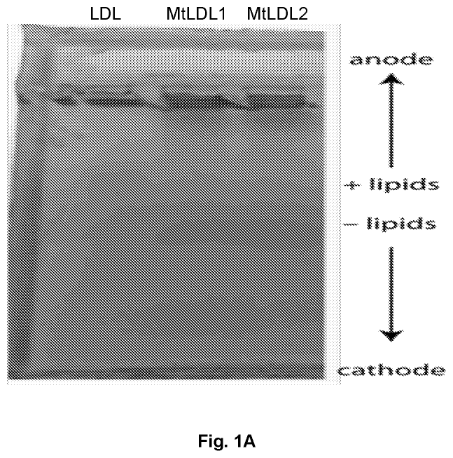

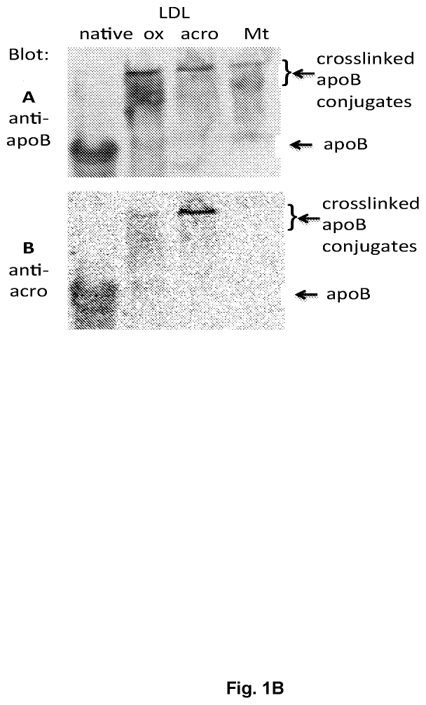

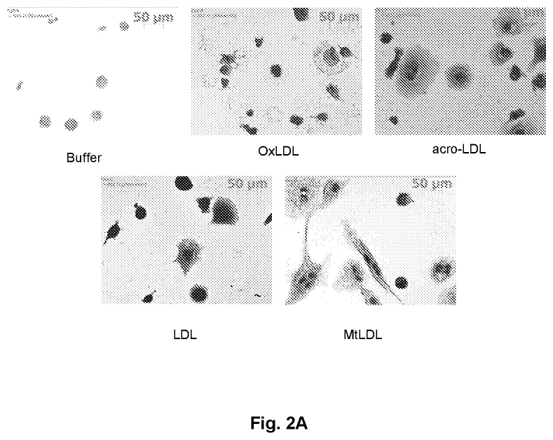

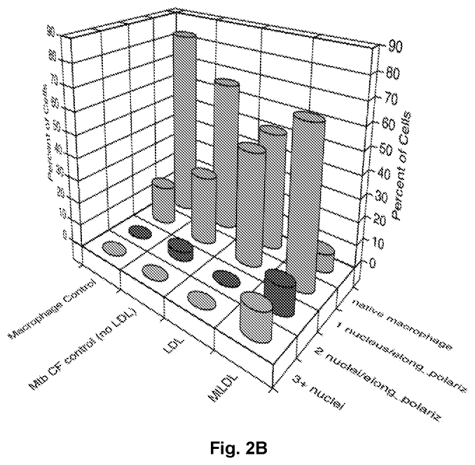

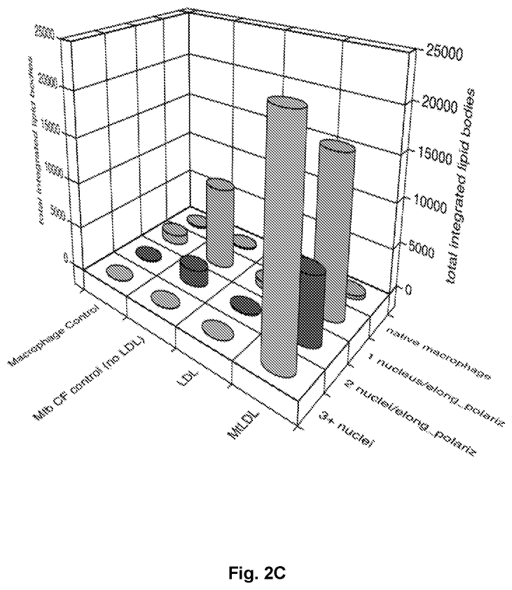

MtLDL is a specific mycobacterial modification of low density lipoprotein (LDL), a host biomolecule. The study characterized MtLDL to assess whether it was analogous to other known forms of modified LDL present in human serum. MtLDL is distinct from known modified LDLs different from other LDL species like oxidized LDL (OxLDL) (Xu, S.; Huang, Y.; Xie, Y.; Lan, T.; Le, K.; Chen, J.; Chen, S.; Gao, S.; Xu, X.; Shen, X. et al. Evaluation of Foam Cell Formation in Cultured Macrophages: An Improved Method with Oil Red O Staining and Dii-Oxldl Uptake. Cytotechnology 2010, 62, 473-481. doi: 10.1007/s10616-010-9290-0) and acrolein-modified LDL (MDA-LDL) present in human blood, that are associated with atherosclerotic plaques (Yoshida, M.; Higashi, K.; Kobayashi, E.; Saeki, N.; Wakui, K.; Kusaka, T.; Takizawa, H.; Kashiwado, K.; Suzuki, N.; Fukuda, K. et al. Correlation between Images of Silent Brain Infarction, Carotid Atherosclerosis and White Matter Hyperintensity, and Plasma Levels of Acrolein, Il-6 and Crp. Atherosclerosis 2010, 211, 475-479. doi: 10.1016/j.atherosclerosis.2010.03.031.) LDL generated from HepG2 human liver cells was incubated with a Mycobacterium tuberculosis (M. tb) strain for CDC1551 or H37Rv for a week to generate MtLDL. The culture filtrates were further washed and concentrated before using the MtLDL for experiments (FIG. 1A). The study described herein shows that MtLDL so generated was larger/more positively charged than native LDL, using agarose gel electrophoresis (0.8% w/v) in barbital buffer followed by staining with Fat Red 7B dye (FIG. 1A). In contrast, OxLDL and MDA-LDL are more negatively charged than native LDL (Watanabe, K.; Nakazato, Y.; Saiki, R.; Igarashi, K.; Kitada, M.; Ishii, I. Acrolein-Conjugated Low-Density Lipoprotein Induces Macrophage Foam Cell Formation. Atherosclerosis 2013, 227, 51-57. doi: 10.1016/j.atherosclerosis.2012.12.020.) The study further shows that MtLDL is recognized by anti-ApoB antibody that recognizes the N-terminus of ApoB-100 (FIG. 1B, Blot A), showing that the ApoB100 N terminus is retained. The study also shows that MtLDL is not recognized by an anti-acrolein antibody that specifically binds MDA-LDL, suggesting that MtLDL contains a modified apolipoprotein B (ApoB) that is not an acrolein derivative (FIG. 1B, Blot B). The study further shows that MtLDL can form a higher level of foamy macrophages (FM) in THP-1 macrophage cultures than other LDL species, e.g., native LDL, oxLDL and MDA-LDL (FIG. 2A). MtLDL induces formation of multinucleated macrophages with a high concentration of lipid bodies compared to native LDL (FIG. 2B-C). This study highlights both physical and functional differences between MtLDL and other LDL species.

### Example 2: Development of Antibodies for Recognizing MtLDL

Specific reagents to detect Mycobacteria modified low density lipoprotein (MtLDL) were developed to assess whether MtLDL is present in serum from patients with TB disease. This study describes preparation of antibodies specific for MtLDL. The study identified human antibodies that bind specifically to MtLDL but not native LDL. Monoclonal antibodies against MtLDL (bivalent Fab with appropriate detection tag) were identified by screening a Human Combinatorial Antibody Library (HuCAL, Bio-Rad AbD Serotec GmbH) comprising Fab fragments of human antibodies, with a diversity of 4.5×10¹⁰ different antibodies. The HuCAL was expressed as a phage display library, wherein every phage particle was tagged with an bivalent Fab fragment such that each phage particle expressed a nucleic acid sequence encoding the protein sequence of the bivalent Fab fragment that was tagged to it. The HuCAL phage library was depleted of antibodies that recognize intact LDL. The depleted library was panned for three rounds of binding, elution, and amplification to isolate antibodies specific for MtLDL that comprised a Glutathione-s-transferase (GST) and a 6 Histidine tag (His6 tag) for enabling detection.

### Example 3: A Method for Detecting MtLDL in a Sample

Using a direct ELISA approach, thirteen (13) selected Fab antibody fragments were identified and produced heterologously from the HuCAL phage display library screen were further screened for determining specificity of MtLDL binding. Eight antibodies showed a specific signal at least 4-fold above native LDL background binding, as shown by a higher signal/background ratio in MtLDL-coated wells as compared to non-specific binding to native LDL (FIG. 3).

All 13 selected Fab antibody fragments were further screened as both capture and detection antibodies in every pairwise combination using a sandwich ELISA assay. Five (5) pairs of Fab antibody fragments were identified that showed a good signal-to-background ratio and, therefore, specific detection of MtLDL. A pair of bivalent antibodies, comprising the bivalent Fab No. 6, corresponding to HCVR SEQ ID NO: 3/LCVR SEQ ID NO: 4, paired with the bivalent Fab No. 8 corresponding to HCVR SEQ ID NO: 5/LCVR SEQ ID NO: 6, demonstrated the most optimum specificity and antigen binding curve for MtLDL, and was further selected for calibration and optimization to assess detection of MtLDL levels in cord blood serum samples spiked with MtLDL (FIG. 4).

Using a Sandwich Enzyme-Linked Immunosorbent Assay (ELISA), the pairwise combination of bivalent Fab No. 6 with bivalent Fab No. 8, showed a specific signal more than 4-fold above the native LDL background, showing a higher signal/background ratio in MtLDL-coated wells as compared to non-specific binding to native LDL. The study established a linear response of the sandwich ELISA using the pairwise combination of bivalent Fab No. 6 with bivalent Fab No. 8, to MtLDL spiked into cord blood serum. MtLDL is specifically detected at >=4 ppm in the presence of serum LDL, which is around 10⁶ ng/mL (FIG. 4).

### Example 4: A Method of Detecting Infection with Mycobacterium tuberculosis (M. tb) in a Subject

A limited number of discarded serum samples from the HIV IMPAACT P1041 trial (Madhi, S. A.; Nachman, S.; Violari, A.; Kim, S.; Cotton, M. F.; Bobat, R.; Jean-Philippe, P.; McSherry, G.; Mitchell, C. Primary Isoniazid Prophylaxis against Tuberculosis in Hiv-Exposed Children. N Engl J Med 2011, 365, 21-31. doi: 10.1056/NEJMoa1011214) (P1041, I. “A Randomized, Double Blind, Placebo Controlled Trial to Determine the Efficacy of Isoniazid (Inh) in Preventing Tuberculosis Disease and Latent Tuberculosis Infection among Infants with Perinatal Exposure to Hiv,” 2014; http://impaactnetwork.org/DocFiles/P1041/P1041V2_11Jul07.pdf Last accessed: 24 Jan. 2018) were assayed for the presence of MtLDL. The P1041 trial enrolled infants exposed to HIV in utero who were screened for Human Immunodeficiency Virus (HIV) and Tuberculosis (TB) infection post-birth. If a patient was found to be, or was believed to be, infected with TB, the patient underwent drug treatment with the standard of care. Patients were enrolled into two arms: HIV uninfected (HIV−) and HIV infected (HIV+). Within each arm, patients were categorized into three groups: no TB, LTBI and TBDIS, based on clinical presentation. Patients without Mycobacterium tuberculosis (M. tb) infection were categorized as no TB; patients with a positive tuberculin skin test but lacking any clinical, radiographic or laboratory evidence of disease caused by M. tb were categorized as latent TB infection (LTBI); and patients presenting clinical, radiographic or laboratory evidence of disease caused by M. tb were categorized as TB disease (TBDis). To assess feasibility, sandwich ELISA assays were performed on 44HIV IMPAACT P1041 serum samples, using only 100 μL and a total assay time was 4.5 hours (FIG. 5A). All 44 HIV− pediatric serum samples consisting of: 6 HIV−/TBDIS, 12 HIV−/LTBI and 26 noTB sera, were tested in duplicate (FIG. 5B). TBDis samples are from children who presented at the time of sample collection with clinical symptoms of TB disease: respiratory illness, failure to gain weight, fever. Some were definitively diagnosed as TB disease and some were classified as probably, but could not definitively be, diagnosed as having TB disease. The LatentTB samples were separated into two categories. Latent TB: samples from children who did not exhibit clinical symptoms of disease at the time of sample collection, but had a household exposure or a positive tuberculin skin test; and LatentTB before converting to TBDIS: samples from children who probably had LatentTB at the time of sample collection, but were later classified as TBDis. The NoTB samples were separated into two categories. NoTB: samples from children who never exhibited clinical symptoms of disease, and who did not have a positive tuberculin skin test or household exposure to TB throughout the study; and NoTB before converting to LatentTB: samples from children who did not have TB at the time of sample collection, but who were later classified as converting to latent TB. Most samples pre and post are from different patients and not multiple timeline samples for a single patient. The study discloses an example retrospective analysis of the P1041 HIV− serum samples for determining the change in the level of MtLDL in serum at different stages of Mtb infection. As shown in FIG. 5B, each data point is the mean for the number of samples indicated in parentheses. Error bars are the standard error of the mean. Samples were collected at different times pre- and post-clinical diagnosis with tuberculosis. As shown herein, FIG. 5C, the box represents the 25% ile-75% ile (25 percentile-75 percentile); the line represents the median, and the whiskers, the 10% ile/90% ile (10 percentile/90 percentile) variation. The no TB median was below the 4 ng/ml detection limit. One outlier in the no TB group was discarded as it was greater than 90% outside the median. The data suggests that a detection of MtLDL in serum can be used to distinguish between TB disease, latent TB and no disease. The study shows that the median serum levels of MtLDL trend higher with infection and disease. The median serum concentration was 15-fold higher in TB disease samples and 6-7-fold higher in latent TB samples compared to no TB samples (FIG. 5B).

As shown herein, there is a clear increase of MtLDL in the serum as TB disease progresses, with a 15-fold increase post TB disease diagnosis above NoTB levels (near detection limit, i.e., background).

At the time of no “TB before converting to latent TB” sample collection, patients have no TB infection. Thus, the MtLDL level is the same as NoTB. At the time of “latent TB before converting to TB disease (TBDis)” collection, patients may have been infected with TB, but they had no clinical signs of disease. Thus, the MtLDL level is similar to levels in LatentTB patients.

“Latent” is a clinical term that infection is likely, but there are no outward manifestations of disease. However, pathologically it represents a spectrum of early disease. Hence, MtLDL levels are expected to vary.

“TBDisease” is a clinical term that covers many different stages of disease. Hence, MtLDL levels are expected to vary.

TABLE 1
Amino acid sequence and DNA sequence of MtLDL antibody.
Amino acid sequences of heavy and light chain Variable region of MtLDL antibody.
Amino acid and DNA sequences of heavy (Fd) chain and light chain of MtLDL
bivalent Fab-Alpkaline Phosphatase- FLAG® tag- His6 tag, fusion antibody.
Constant domains CH1 and CL sequence are in italics, dimerization domain
sequence (AP) is double underlined, linker sequences are in bold, FLAG®
tag is underlined with dashed line and His6 tag is in underlined with thick line.
Variable heavy region (SEQ ID NO:1)
QVQLVQSGAEVKKPGASVKVSCKASGYTFTSYYINWVRQAPGQGLEWMGYINPYNGNTRYAQKF
QGRVTMTRDTSISTAYMELSRLRSEDTAVYYCARGMLFAHWGQGTLVTVSS
Variable light region (SEQ ID NO:2)
DIVLTQSPSSLSLSPGERATLSCRASQRVSFNYLAWYQQKPGQAPRLLIYGASKRATGIPARES
GSGSGTDFTLTISSLEPEDFAVYYCMQYLSTPRTFGQGTKVEIKRT
Variable heavy region (SEQ ID NO:3)
EVQLLESGGGLVQPGGSLRLSCAASGFTFRGYYMSWVRQAPGKGLEWVSSISGFSSNTYYADSV
KGRFTISRDNSKNTLYLQMNSLRAEDTAVYYCARVRYLAYAFDYWGQGTLVTVSS
Variable light region (SEQ ID NO:4)
DIELTQPPSVSVSPGQTASITCSGDSLPDKRAYWYQQKPGQAPVLVIYGDSHRPSGIPERFSGS
NSGNTATLTISGTQAEDEADYYCSSWGSRTWVFGGGTKLTVLGQ
Variable heavy region (SEQ ID NO:5)
QVQLVQSGAEVKKPGSSVKVSCKASGGTFSGYYISWVRQAPGQGLEWMGGIIPISGRANYAQKF
QGRVTITADESTSTAYMELSSLRSEDTAVYYCARSRSYYHFDLWGQGTLVTVSS
Variable light region (SEQ ID NO:6)
DIALTQPASVSGSPGQSITISCTGTSSDVGRYNSVSWYQQHPGKAPKLMIYRVSKRPSGVSNRF
SGSKSGNTASLTISGLQAEDEADYYCQSWASLSNVVFGGGTKLTVLGQ
Variable heavy region (SEQ ID NO:7)
EVQLVQSGAEVKKPGESLKISCKGSGYSFTGYVIHWVRQMPGKGLEWMGRIDPSKSYTRYSPSF
QGQVTISADKSISTAYLQWSSLKASDTAMYYCARGLYSGYFDIWGQGTLVTVSS
Variable light region (SEQ ID NO:8)
DIELTQPPSVSVSPGQTASITCSGDALGSKYVHWYQQKPGQAPVLVIYAKNNRPSGIPERFSGS
NSGNTATLTISGTQAEDEADYYCQSRASGIFGRVFGGGTKLTVLGQ
SEQ ID NO.: 9
Format: Fab-A-FH (bivalent Fab-bacterial alkaline phosphatase fusion antibody
followed by FLAG® and His6-tag) VH1B (heavy chain) κ3 (light chain)
Amino acid sequence of Fd chain and tags: Artificial Human Antibody comprising:
Heavy Chain Variable Region; part of Heavy Chain Constant Region; Dimerization
Domain Sequence (AP) position 220-671:
Flag tag position 672-679, and HiS6 tag position 683-688.
QVQLVQSGAEVKKPGASVKVSCKASGYTFTSYYINWVRQAPGQGLEWMGYINPYNGNTRYAQKFQGRVTM
TRDTSISTAYMELSRLRSEDTAVYYCARGMLFAHWGQGTLVTVSSASTKGPSVFPLAPSSKSTSGGTAAL
GCLVKDYFPEPVTVSWNSGALTSGVHTFPAVLOSSGLYSLSSVVTVPSSSLGTQTYICNVNHKPSNTKVD
KKVEPKSEFKAEMPVLENRAAQGDITTPGGARRLTGDQTAALRDSLSDKPAKNIILLIGDGMGDSEITAA
RNYAEGAGGFFKGIDALPLTGQYTHYALNRKTGKPDYVTSSAASATAWSTGVKTYNGALGVDIHEKDHPT
ILEMAKAAGLATGNVSTAELQDATPAALVAHVTSRKCYGPSATSEKCPGNALEKGGKGSITEQLLNARAD
VTLGGGAKTFAETATAGEWQGKTLREQAQARGYQLVSDAASLNSVTEANQQKPLLGLFADGNMPVRWLGP
KATYHGNIDKPAVTCTPNPORNDSVPTLAQMTDKAIELLSKNEKGFFLQVEGASIDKQDHAANPCGQIGE
TVDLDEAVQRALEFAKKEGNTLVIVTADHAHASQIVAPDTKAPGLTQALNTKDGAVMVMSYGNSEEDSQE

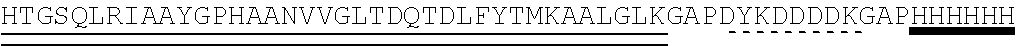

SEQ ID NO.: 10: Amino acid sequence of light chain Artificial Human
Antibody comprising: Light Chain Variable Region and Light Chain
Constant Region
DIVLTQSPSSLSLSPGERATLSCRASQRVSFNYLAWYQQKPGQAPRLLIYGASKRATGIPARFSGSGSGT
DFTLTISSLEPEDFAVYYCMQYLSTPRTFGQGTKVEIKRTVAAPSVF/FPPSDEQLKSGTASVVCLLNNF
YPREAKVQPIKVDNALQSGNSQESVTEQDSKDSTYSLESTLTLSKADYEKHKVITACEVTHQGLESPVTKSF
NRGEA
SEQ ID NO.: 11: DNA sequences of Fd chain with tags
CAGGTGCAATTGGTGCAGAGCGGTGCGGAAGTGAAAAAACCGGGTGCCAGCGTGAAAGTTAGCTGCAAAG
CGTCCGGATATACCTTCACTTCTTACTACATCAACTGGGTGCGCCAGGCCCCGGGCCAGGGCCTCGAGTG
GATGGGCTACATCAACCCGTACAACGGCAACACGCGTTACGCGCAGAAATTTCAGGGCCGGGTGACCATG
ACCCGTGATACCAGCATTAGCACCGCGTATATGGAACTGAGCCGTCTGCGTAGCGAAGATACGGCCGTGT
ATTATTGCGCGCGTGGTATGCTGTTCGCTCATTGGGGCCAAGGCACCCTGGTGACTGTTAGCTCAGCGTC
GACCAAAGGCCCGAGCGTGTTTCCGCTGGCCCCGAGCAGCAAAAGCACCAGCGGCGGCACCGCCGCACTG
GGCTGCCTGGTGAAAGATTATTTCCCGGAACCAGTGACCGTGAGCTGGAACAGCGGTGCCCTGACCAGCG
GCGTGCATACCTTTCCGGCGGTGCTGCAAAGCAGCGGCCTGTATAGCCTGAGCAGCGTTGTGACCGTGCC
GAGCAGCAGCCTGGGCACCCAGACCTATATTTGCAACGTCAACCATAAACCGAGCAACACCAAAGTCGAT
AAAAAAGTCGAACCGAAAAGCGAATTCAAGGCTGAAATGCCTGTTCTGGAAAACCGGGCTGCTCAGGGCG
ATATTACTACACCCGGCGGTGCTCGCCGTTTAACGGGTGATCAGACTGCCGCTCTGCGTGATTCTCTTAG
CGATAAACCTGCAAAAAATATTATTTTGCTGATTGGCGATGGGATGGGGGACTCGGAAATTACTGCCGCA
CGTAATTATGCCGAAGGTGCGGGCGGCTTTTTTAAAGGTATAGATGCCTTACCGCTTACCGGGCAATACA
CTCACTATGCGCTGAATAGAAAAACCGGCAAACCGGACTACGTCACCAGCTCGGCTGCATCAGCAACCGC
CTGGTCAACCGGTGTCAAAACCTATAACGGCGCGCTGGGCGTCGATATTCACGAAAAAGATCACCCAACG
ATTCTGGAAATGGCAAAAGCCGCAGGTCTGGCGACCGGTAACGTTTCTACCGCAGAGTTGCAGGATGCCA
CGCCCGCTGCGCTGGTGGCACATGTGACCTCGCGCAAATGCTACGGTCCGAGCGCGACCAGTGAAAAATG
TCCGGGTAACGCTCTGGAAAAAGGCGGAAAAGGATCGATTACCGAACAGCTGCTTAACGCTCGTGCCGAC
GTTACGCTTGGCGGCGGCGCAAAAACCTTTGCTGAAACGGCAACCGCTGGTGAATGGCAGGGAAAAACGC
TGCGTGAACAGGCACAGGCGCGTGGTTATCAGTTGGTGAGCGATGCTGCCTCACTGAACTCGGTGACGGA
AGCGAATCAGCAAAAACCCCTGCTTGGCCTGTTTGCTGACGGCAATATGCCAGTGCGCTGGCTAGGACCG
AAAGCAACGTACCATGGCAATATCGATAAGCCCGCAGTCACCTGTACGCCAAATCCGCAACGTAATGACA
GTGTACCAACCCTGGCGCAGATGACCGACAAAGCCATTGAATTGTTGAGTAAAAATGAGAAAGGCTTTTT
CCTGCAAGTTGAAGGTGCGTCAATCGATAAACAGGATCATGCTGCGAATCCTTGTGGGCAAATTGGCGAG
ACGGTCGATCTCGATGAAGCCGTACAACGGGCGCTGGAGTTCGCTAAAAAGGAGGGTAACACGCTGGTCA
TAGTCACCGCTGATCACGCCCACGCCAGCCAGATTGTTGCGCCGGATACCAAAGCTCCGGGCCTCACCCA
GGCGCTAAATACCAAAGATGGCGCAGTGATGGTGATGAGTTACGGGAACTCCGAAGAGGATTCACAAGAA
CATACCGGCAGTCAGTTGCGTATTGCGGCGTATGGCCCGCATGCCGCCAATGTTGTTGGACTGACCGACC
AGACCGATCTCTTCTACACCATGAAAGCCGCTCTGGGGCTGAAAGGCGCGCCGGACTATAAAGATGACGA
TGACAAAGGCGCGCCGCACCATCATCACCATCAC
SEQ ID NO.: 12: DNA sequence of Light chain Artificial Human Antibody
comprising: DNA sequence of Light Chain Variable Region and Light
Chain Constant Region
GATATCGTGCTGACCCAGAGCCCGAGCAGCCTGAGCCTGAGCCCGGGTGAACGTGCCACCCTGAGCTGCA
GAGCGAGCCAGCGTGTTTCTTTCAACTACCTGGCTTGGTACCAGCAGAAACCGGGCCAGGCCCCGCGTCT
ATTAATCTACGGTGCTTCTAAACGTGCGACCGGCATTCCGGCGCGTTTTAGCGGCAGCGGATCCGGCACC
GATTTCACCCTGACCATTAGCAGCCTGGAACCGGAAGACTTTGCGGTGTATTATTGCATGCAGTACCTGT
CTACTCCGCGTACCTTTGGCCAGGGCACGAAAGTTGAAATTAAACGTACGGTGGCCGCACCGAGCGTGTT
TATCTTTCCGCCGAGCGATGAACAGCTGAAAAGCGGCACCGCCAGCGTGGTGTGCCTGCTGAACAACTTT
TATCCGCGCGAAGCCAAAGTGCAGTGGAAAGTGGATAACGCCCTGCAAAGCGGCAACAGCCAGGAAAGCG
TTACCGAACAGGATAGCAAAGATAGCACCTACAGCCTGAGCAGCACCCTGACCCTGAGCAAAGCCGATTA
TGAAAAACATAAAGTGTATGCCTGCGAAGTGACCCATCAGGGCCTGAGCAGCCCAGTGACCAAAAGTTTT
AACCGCGGCGAGGCC
SEQ ID NO.: 13
Format: Fab-A-FH (bivalent Fab-bacterial alkaline phosphatase fusion antibody
followed by FLAG® and His6-tag) VH3-23 (heavy chain) λ3 (light chain)
Amino acid sequence of Fd chain and tags Artificial Human Antibody comprising:
Heavy Chain Variable Region; part of Heavy Chain Constant Region; Dimerization
Domain Sequence (AP) position 223-672: Flag tag position 676-683, and HiS6
tag position 687-692.
EVQLLESGGGLVQPGGSLRLSCAASGFTFRGYYMSWVRQAPGKGLEWVSSISGFSSNTYYADSVKGRFTI
SRDNSKNTLYLQMNSLRAEDTAVYYCARVRYLAYAFDYWGQGTLVTVSSASTKGPSVFPLAPSSKSTSGG
TAALGCLVKDYFPEPVTVSWNSGALTSGWITFPAVLOSSGLYSLSSVVTVPSSSLGTOTYICNVATIIKPSN
TKVDKKVEPKSEFKAEMPVLENRAAQGDITTPGGARRLTGDQTAALRDSLSDKPAKNIILLIGDGMGDSE
ITAARNYAEGAGGFFKGIDALPLTGQYTHYALNRKTGKPDYVTSSAASATAWSTGVKTYNGALGVDIHEK
DHPTILEMAKAAGLATGNVSTAELQDATPAALVAHVTSRKCYGPSATSEKCPGNALEKGGKGSITEQLLN
ARADVTLGGGAKTFAETATAGEWQGKTLREQAQARGYQLVSDAASLNSVTEANQQKPLLGLFADGNMPVR
WLGPKATYHGNIDKPAVTCTPNPQRNDSVPTLAQMTDKAIELLSKNEKGFFLQVEGASIDKQDHAANPCG
QIGETVDLDEAVQRALEFAKKEGNTLVIVTADHAHASQIVAPDTKAPGLTQALNTKDGAVMVMSYGNSEE

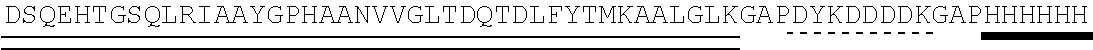

SEQ ID NO.: 14: AMINO ACID SEQUENCE OF LIGHT CHAIN Artificial Human
Antibody comprising: Light Chain Variable Region and Light Chain
Constant Region
DIELTQPPSVSVSPGQTASITCSGDSLPDKRAYWYQQKPGQAPVLVIYGDSHRPSGIPERFSGSNSGNTA
TLTISGTQAEDEADYYCSSWGSRTWVFGGGTKLTVLGQPKAAPSVTLFPPSSEELQANKATLVCLISDFY
PGAVTVAWKADSSPVKAGVETTTPSKOSNNKYAASSYLSLTPEOWKSERSYSCOVITIEGSTVEKTVAPTE
A
SEQ ID NO.: 15: DNA SEQUENCES OF Fd chain with tags
GAAGTGCAATTGCTGGAAAGCGGCGGTGGCCTGGTGCAGCCGGGTGGCAGCCTGCGTCTGAGCTGCGCGG
CGTCCGGATTCACCTTTCGTGGTTACTACATGTCTTGGGTGCGCCAGGCCCCGGGCAAAGGTCTCGAGTG
GGTTTCCTCTATCTCTGGTTTCTCTTCTAACACCTACTATGCGGATAGCGTGAAAGGCCGCTTTACCATC
AGCCGCGATAATTCGAAAAACACCCTGTATCTGCAAATGAACAGCCTGCGTGCGGAAGATACGGCCGTGT
ATTATTGCGCGCGTGTTCGTTACCTGGCTTACGCTTTCGATTACTGGGGCCAAGGCACCCTGGTGACTGT
TAGCTCAGCGTCGACCAAAGGCCCGAGCGTGTTTCCGCTGGCCCCGAGCAGCAAAAGCACCAGCGGCGGC
ACCGCCGCACTGGGCTGCCTGGTGAAAGATTATTTCCCGGAACCAGTGACCGTGAGCTGGAACAGCGGTG
CCCTGACCAGCGGCGTGCATACCTTTCCGGCGGTGCTGCAAAGCAGCGGCCTGTATAGCCTGAGCAGCGT
TGTGACCGTGCCGAGCAGCAGCCTGGGCACCCAGACCTATATTTGCAACGTCAACCATAAACCGAGCAAC
ACCAAAGTCGATAAAAAAGTCGAACCGAAAAGCGAATTCAAGGCTGAAATGCCTGTTCTGGAAAACCGGG
CTGCTCAGGGCGATATTACTACACCCGGCGGTGCTCGCCGTTTAACGGGTGATCAGACTGCCGCTCTGCG
TGATTCTCTTAGCGATAAACCTGCAAAAAATATTATTTTGCTGATTGGCGATGGGATGGGGGACTCGGAA
ATTACTGCCGCACGTAATTATGCCGAAGGTGCGGGCGGCTTTTTTAAAGGTATAGATGCCTTACCGCTTA
CCGGGCAATACACTCACTATGCGCTGAATAGAAAAACCGGCAAACCGGACTACGTCACCAGCTCGGCTGC
ATCAGCAACCGCCTGGTCAACCGGTGTCAAAACCTATAACGGCGCGCTGGGCGTCGATATTCACGAAAAA
GATCACCCAACGATTCTGGAAATGGCAAAAGCCGCAGGTCTGGCGACCGGTAACGTTTCTACCGCAGAGT
TGCAGGATGCCACGCCCGCTGCGCTGGTGGCACATGTGACCTCGCGCAAATGCTACGGTCCGAGCGCGAC
CAGTGAAAAATGTCCGGGTAACGCTCTGGAAAAAGGCGGAAAAGGATCGATTACCGAACAGCTGCTTAAC
GCTCGTGCCGACGTTACGCTTGGCGGCGGCGCAAAAACCTTTGCTGAAACGGCAACCGCTGGTGAATGGC
AGGGAAAAACGCTGCGTGAACAGGCACAGGCGCGTGGTTATCAGTTGGTGAGCGATGCTGCCTCACTGAA
CTCGGTGACGGAAGCGAATCAGCAAAAACCCCTGCTTGGCCTGTTTGCTGACGGCAATATGCCAGTGCGC
TGGCTAGGACCGAAAGCAACGTACCATGGCAATATCGATAAGCCCGCAGTCACCTGTACGCCAAATCCGC
AACGTAATGACAGTGTACCAACCCTGGCGCAGATGACCGACAAAGCCATTGAATTGTTGAGTAAAAATGA
GAAAGGCTTTTTCCTGCAAGTTGAAGGTGCGTCAATCGATAAACAGGATCATGCTGCGAATCCTTGTGGG
CAAATTGGCGAGACGGTCGATCTCGATGAAGCCGTACAACGGGCGCTGGAGTTCGCTAAAAAGGAGGGTA
ACACGCTGGTCATAGTCACCGCTGATCACGCCCACGCCAGCCAGATTGTTGCGCCGGATACCAAAGCTCC
GGGCCTCACCCAGGCGCTAAATACCAAAGATGGCGCAGTGATGGTGATGAGTTACGGGAACTCCGAAGAG
GATTCACAAGAACATACCGGCAGTCAGTTGCGTATTGCGGCGTATGGCCCGCATGCCGCCAATGTTGTTG
GACTGACCGACCAGACCGATCTCTTCTACACCATGAAAGCCGCTCTGGGGCTGAAAGGCGCGCCGGACTA
TAAAGATGACGATGACAAAGGCGCGCCGCACCATCATCACCATCAC
SEQ ID NO.: 16: DNA sequence of Light chain Artificial Human Antibody
comprising: Light Chain Variable Region and Light Chain Constant
Region
GATATCGAACTGACCCAGCCGCCGAGCGTGAGCGTGAGCCCGGGCCAGACCGCGAGCATTACCTGTAGCG
GCGATTCTCTGCCGGACAAACGTGCTTACTGGTACCAGCAGAAACCGGGCCAGGCGCCGGTGCTGGTGAT
CTACGGTGACTCTCATCGTCCGAGCGGCATCCCGGAACGTTTTAGCGGATCCAACAGCGGCAACACCGCG
ACCCTGACCATTAGCGGCACCCAGGCGGAAGACGAAGCGGATTATTACTGCTCTTCTTGGGGTTCTCGTA
CTTGGGTGTTTGGCGGCGGCACGAAGTTAACCGTTCTTGGCCAGCCGAAAGCCGCCCCAAGCGTGACCCT
GTTTCCGCCGAGCAGCGAAGAACTGCAAGCCAACAAAGCCACCCTGGTTTGCCTGATCAGCGATTTTTAT
CCGGGTGCCGTGACCGTGGCCTGGAAAGCCGATAGCAGCCCGGTGAAAGCCGGCGTGGAAACCACCACCC
CGAGCAAACAGAGCAACAACAAATATGCCGCCAGCAGCTATCTGAGCCTGACCCCGGAACAGTGGAAAAG
CCATCGCAGCTATAGTTGTCAAGTGACCCATGAAGGCAGCACCGTGGAAAAAACCGTGGCCCCGACCGAG
GCC
SEQ ID NO.: 17
Format: Fab-A-FH (bivalent Fab-bacterial alkaline phosphatase fusion
antibody followed by FLAG® and His6-tag) VH1A (heavy chain) and λ2
(light chain)
Amino acid sequence of Fd chain and tags Artificial Human Antibody
comprising: Heavy Chain Variable Region; part of Heavy Chain Constant
Region; Dimerization Domain Sequence (AP) position 222-671: Flag tag
position 675-682, and HiS6 tag position 686-691.
QVQLVQSGAEVKKPGSSVKVSCKASGGTFSGYYISWVRQAPGQGLEWMGGIIPISGRANYAQKFQGRVTI
TADESTSTAYMELSSLRSEDTAVYYCARSRSYYHFDLWGQGTLVTVSSASTKGPSVFPLAPSSKSTSGGT
AALGCLVKDYFPEPVTIISWNSGALTSGVHTFPAVLOSSGLYSLESVVTVPSSELGTOTYICNVMHKPENT
KVDKKVEPKSEFKAEMPVLENRAAQGDITTPGGARRLTGDQTAALRDSLSDKPAKNIILLIGDGMGDSEI
TAARNYAEGAGGFFKGIDALPLTGQYTHYALNRKTGKPDYVTSSAASATAWSTGVKTYNGALGVDIHEKD
HPTILEMAKAAGLATGNVSTAELQDATPAALVAHVTSRKCYGPSATSEKCPGNALEKGGKGSITEQLLNA
RADVTLGGGAKTFAETATAGEWQGKTLREQAQARGYQLVSDAASLNSVTEANQQKPLLGLFADGNMPVRW
LGPKATYHGNIDKPAVTCTPNPQRNDSVPTLAQMTDKAIELLSKNEKGFFLQVEGASIDKQDHAANPCGQ
IGETVDLDEAVQRALEFAKKEGNTLVIVTADHAHASQIVAPDTKAPGLTQALNTKDGAVMVMSYGNSEED

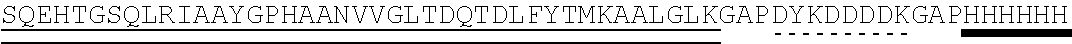

SEQ ID NO.: 18: Amino acid sequence of light chain Artificial Human
Antibody comprising: Light Chain Variable Region and Light Chain
Constant Region
DIALTQPASVSGSPGQSITISCTGTSSDVGRYNSVSWYQQHPGKAPKLMIYRVSKRPSGVSNRFSGSKSG
NTASLTISGLQAEDEADYYCQSWASLSNVVFGGGTKLTVLGQPKAAPSVTLFPPSSEELOANKATLVCLI
SDFYPGAVTVAWKADESPVKAGVETTTPSKOSNNKYAASSYLELTPEOWKSERSYSCOVTHEGSTVEKTV
APTEA
SEQ ID NO.: 19: DNA sequences of Fd chain with tags
CAGGTGCAATTGGTGCAGAGCGGTGCCGAAGTGAAAAAACCGGGCAGCAGCGTGAAAGTTAGCTGCAAAG
CATCCGGAGGGACGTTTTCTGGTTACTACATCTCTTGGGTGCGCCAGGCCCCGGGCCAGGGCCTCGAGTG
GATGGGCGGTATCATCCCGATCTCTGGCCGTGCGAACTACGCCCAGAAATTTCAGGGCCGGGTGACCATT
ACCGCCGATGAAAGCACCAGCACCGCCTATATGGAACTGAGCAGCCTGCGCAGCGAAGATACGGCCGTGT
ATTATTGCGCGCGTTCTCGTTCTTACTACCATTTCGATCTGTGGGGCCAAGGCACCCTGGTGACTGTTAG
CTCAGCGTCGACCAAAGGCCCGAGCGTGTTTCCGCTGGCCCCGAGCAGCAAAAGCACCAGCGGCGGCACC
GCCGCACTGGGCTGCCTGGTGAAAGATTATTTCCCGGAACCAGTGACCGTGAGCTGGAACAGCGGTGCCC
TGACCAGCGGCGTGCATACCTTTCCGGCGGTGCTGCAAAGCAGCGGCCTGTATAGCCTGAGCAGCGTTGT
GACCGTGCCGAGCAGCAGCCTGGGCACCCAGACCTATATTTGCAACGTCAACCATAAACCGAGCAACACC
AAAGTCGATAAAAAAGTCGAACCGAAAAGCGAATTCAAGGCTGAAATGCCTGTTCTGGAAAACCGGGCTG
CTCAGGGCGATATTACTACACCCGGCGGTGCTCGCCGTTTAACGGGTGATCAGACTGCCGCTCTGCGTGA
TTCTCTTAGCGATAAACCTGCAAAAAATATTATTTTGCTGATTGGCGATGGGATGGGGGACTCGGAAATT
ACTGCCGCACGTAATTATGCCGAAGGTGCGGGCGGCTTTTTTAAAGGTATAGATGCCTTACCGCTTACCG
GGCAATACACTCACTATGCGCTGAATAGAAAAACCGGCAAACCGGACTACGTCACCAGCTCGGCTGCATC
AGCAACCGCCTGGTCAACCGGTGTCAAAACCTATAACGGCGCGCTGGGCGTCGATATTCACGAAAAAGAT
CACCCAACGATTCTGGAAATGGCAAAAGCCGCAGGTCTGGCGACCGGTAACGTTTCTACCGCAGAGTTGC
AGGATGCCACGCCCGCTGCGCTGGTGGCACATGTGACCTCGCGCAAATGCTACGGTCCGAGCGCGACCAG
TGAAAAATGTCCGGGTAACGCTCTGGAAAAAGGCGGAAAAGGATCGATTACCGAACAGCTGCTTAACGCT
CGTGCCGACGTTACGCTTGGCGGCGGCGCAAAAACCTTTGCTGAAACGGCAACCGCTGGTGAATGGCAGG
GAAAAACGCTGCGTGAACAGGCACAGGCGCGTGGTTATCAGTTGGTGAGCGATGCTGCCTCACTGAACTC
GGTGACGGAAGCGAATCAGCAAAAACCCCTGCTTGGCCTGTTTGCTGACGGCAATATGCCAGTGCGCTGG
CTAGGACCGAAAGCAACGTACCATGGCAATATCGATAAGCCCGCAGTCACCTGTACGCCAAATCCGCAAC
GTAATGACAGTGTACCAACCCTGGCGCAGATGACCGACAAAGCCATTGAATTGTTGAGTAAAAATGAGAA
AGGCTTTTTCCTGCAAGTTGAAGGTGCGTCAATCGATAAACAGGATCATGCTGCGAATCCTTGTGGGCAA
ATTGGCGAGACGGTCGATCTCGATGAAGCCGTACAACGGGCGCTGGAGTTCGCTAAAAAGGAGGGTAACA
CGCTGGTCATAGTCACCGCTGATCACGCCCACGCCAGCCAGATTGTTGCGCCGGATACCAAAGCTCCGGG
CCTCACCCAGGCGCTAAATACCAAAGATGGCGCAGTGATGGTGATGAGTTACGGGAACTCCGAAGAGGAT
TCACAAGAACATACCGGCAGTCAGTTGCGTATTGCGGCGTATGGCCCGCATGCCGCCAATGTTGTTGGAC
TGACCGACCAGACCGATCTCTTCTACACCATGAAAGCCGCTCTGGGGCTGAAAGGCGCGCCGGACTATAA
AGATGACGATGACAAAGGCGCGCCGCACCATCATCACCATCAC
SEQ ID NO.: 20: DNA sequence of Light chain Artificial Human Antibody
comprising: Light Chain Variable Region and Light Chain Constant
Region
GATATCGCGCTGACCCAGCCGGCGAGCGTGAGCGGTAGCCCGGGCCAGAGCATTACCATTAGCTGCACCG
GCACCAGCAGCGATGTGGGCCGTTACAACTCTGTGTCTTGGTACCAGCAGCATCCGGGCAAGGCGCCGAA
ACTGATGATCTACCGTGTTTCTAAACGTCCGAGCGGCGTGAGCAACCGTTTTAGCGGATCCAAAAGCGGC
AACACCGCGAGCCTGACCATTAGCGGCCTGCAAGCGGAAGACGAAGCGGATTATTACTGCCAGTCTTGGG
CTTCTCTGTCTAACGTTGTGTTTGGCGGCGGCACGAAGTTAACCGTTCTTGGCCAGCCGAAAGCCGCCCC
AAGCGTGACCCTGTTTCCGCCGAGCAGCGAAGAACTGCAAGCCAACAAAGCCACCCTGGTTTGCCTGATC
AGCGATTTTTATCCGGGTGCCGTGACCGTGGCCTGGAAAGCCGATAGCAGCCCGGTGAAAGCCGGCGTGG
AAACCACCACCCCGAGCAAACAGAGCAACAACAAATATGCCGCCAGCAGCTATCTGAGCCTGACCCCGGA
ACAGTGGAAAAGCCATCGCAGCTATAGTTGTCAAGTGACCCATGAAGGCAGCACCGTGGAAAAAACCGTG
GCCCCGACCGAGGCC
SEQ ID NO.: 21
Format: Fab-A-FH (bivalent Fab-bacterial alkaline phosphatase fusion
antibody followed by FLAG® and His6-tag)
Variable domain subfamilies: VH5 (heavy chain) and λ3 (light chain)
Amino acid sequence of Fd chain and tags Artificial Human Antibody
comprising: Heavy Chain Variable Region; part of Heavy Chain Constant
Region; Dimerization Domain Sequence (AP) position 223-671: Flag tag
position 675-682, and HiS6 tag position 686-691.
EVQLVQSGAEVKKPGESLKISCKGSGYSFTGYVIHWVRQMPGKGLEWMGRIDPSKSYTRYSPSFQGQVTI
SADKSISTAYLQWSSLKASDTAMYYCARGLYSGYFDIWGQGTLVTVSSASTKGPSVFPLAPSSKSTSGGT
AALGCLVKDYFPEPVTVSWNSGALTSGVHTFPAVLOSSGLYSLSSVVTVPSSSLGTOTYICNVMHKPSNT
KVDKKVEPKSEFKAEMPVLENRAAQGDITTPGGARRLTGDQTAALRDSLSDKPAKNIILLIGDGMGDSEI
TAARNYAEGAGGFFKGIDALPLTGQYTHYALNRKTGKPDYVTSSAASATAWSTGVKTYNGALGVDIHEKD
HPTILEMAKAAGLATGNVSTAELQDATPAALVAHVTSRKCYGPSATSEKCPGNALEKGGKGSITEQLLNA
RADVTLGGGAKTFAETATAGEWQGKTLREQAQARGYQLVSDAASLNSVTEANQQKPLLGLFADGNMPVRW
LGPKATYHGNIDKPAVTCTPNPORNDSVPTLAQMTDKAIELLSKNEKGFFLOVEGASIDKODHAANPCGQ
IGETVDLDEAVORALEFAKKEGNTLVIVTADHAHASOIVAPDTKAPGLTOALNTKDGAVMVMSYGNSEED

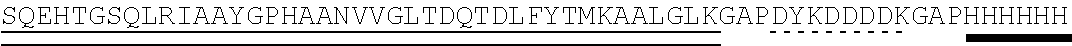

SEQ ID NO.: 22: Amino acid sequence of light chain Artificial Human
Antibody comprising: Light Chain Variable Region and Light Chain
Constant Region
dieltqppsysyspgqtasitcsgdalgskyvhwyqqkpgqapvlviyaknnrpsgiperfsgsnsgnta
tltisgtqaedeadyycgsrasgifgrvfgggtkltvlgqpkaapsvtlfppsseelgankativclisd
fypgavtvawkadsspvkagvetttpskgsnnkyaassylsltpeqwkshrsyscqvthegstvektvap
tea
SEQ ID NO.: 23: DNA sequences of Fd chain with tags
GAAGTGCAATTGGTGCAGAGCGGTGCGGAAGTGAAAAAACCGGGCGAAAGCCTGAAAATTAGCTGCAAAG
GCTCCGGATATAGCTTCACTGGTTACGTTATCCATTGGGTGCGCCAGATGCCGGGCAAAGGTCTCGAGTG
GATGGGCCGTATCGACCCGTCTAAAAGCTACACCCGTTATAGCCCGAGCTTTCAGGGCCAGGTGACCATT
AGCGCGGATAAAAGCATCAGCACCGCGTATCTGCAATGGAGCAGCCTGAAAGCGAGCGATACCGCGATGT
ATTATTGCGCGCGTGGTCTGTACTCTGGTTACTTCGATATCTGGGGCCAAGGCACCCTGGTGACTGTTAG
CTCAGCGTCGACCAAAGGCCCGAGCGTGTTTCCGCTGGCCCCGAGCAGCAAAAGCACCAGCGGCGGCACC
GCCGCACTGGGCTGCCTGGTGAAAGATTATTTCCCGGAACCAGTGACCGTGAGCTGGAACAGCGGTGCCC
TGACCAGCGGCGTGCATACCTTTCCGGCGGTGCTGCAAAGCAGCGGCCTGTATAGCCTGAGCAGCGTTGT
GACCGTGCCGAGCAGCAGCCTGGGCACCCAGACCTATATTTGCAACGTCAACCATAAACCGAGCAACACC
AAAGTCGATAAAAAAGTCGAACCGAAAAGCGAATTCAAGGCTGAAATGCCTGTTCTGGAAAACCGGGCTG
CTCAGGGCGATATTACTACACCCGGCGGTGCTCGCCGTTTAACGGGTGATCAGACTGCCGCTCTGCGTGA
TTCTCTTAGCGATAAACCTGCAAAAAATATTATTTTGCTGATTGGCGATGGGATGGGGGACTCGGAAATT
ACTGCCGCACGTAATTATGCCGAAGGTGCGGGCGGCTTTTTTAAAGGTATAGATGCCTTACCGCTTACCG
GGCAATACACTCACTATGCGCTGAATAGAAAAACCGGCAAACCGGACTACGTCACCAGCTCGGCTGCATC
AGCAACCGCCTGGTCAACCGGTGTCAAAACCTATAACGGCGCGCTGGGCGTCGATATTCACGAAAAAGAT
CACCCAACGATTCTGGAAATGGCAAAAGCCGCAGGTCTGGCGACCGGTAACGTTTCTACCGCAGAGTTGC
AGGATGCCACGCCCGCTGCGCTGGTGGCACATGTGACCTCGCGCAAATGCTACGGTCCGAGCGCGACCAG
TGAAAAATGTCCGGGTAACGCTCTGGAAAAAGGCGGAAAAGGATCGATTACCGAACAGCTGCTTAACGCT
CGTGCCGACGTTACGCTTGGCGGCGGCGCAAAAACCTTTGCTGAAACGGCAACCGCTGGTGAATGGCAGG
GAAAAACGCTGCGTGAACAGGCACAGGCGCGTGGTTATCAGTTGGTGAGCGATGCTGCCTCACTGAACTC
GGTGACGGAAGCGAATCAGCAAAAACCCCTGCTTGGCCTGTTTGCTGACGGCAATATGCCAGTGCGCTGG
CTAGGACCGAAAGCAACGTACCATGGCAATATCGATAAGCCCGCAGTCACCTGTACGCCAAATCCGCAAC
GTAATGACAGTGTACCAACCCTGGCGCAGATGACCGACAAAGCCATTGAATTGTTGAGTAAAAATGAGAA
AGGCTTTTTCCTGCAAGTTGAAGGTGCGTCAATCGATAAACAGGATCATGCTGCGAATCCTTGTGGGCAA
ATTGGCGAGACGGTCGATCTCGATGAAGCCGTACAACGGGCGCTGGAGTTCGCTAAAAAGGAGGGTAACA
CGCTGGTCATAGTCACCGCTGATCACGCCCACGCCAGCCAGATTGTTGCGCCGGATACCAAAGCTCCGGG
CCTCACCCAGGCGCTAAATACCAAAGATGGCGCAGTGATGGTGATGAGTTACGGGAACTCCGAAGAGGAT
TCACAAGAACATACCGGCAGTCAGTTGCGTATTGCGGCGTATGGCCCGCATGCCGCCAATGTTGTTGGAC
TGACCGACCAGACCGATCTCTTCTACACCATGAAAGCCGCTCTGGGGCTGAAAGGCGCGCCGGACTATAA
AGATGACGATGACAAAGGCGCGCCGCACCATCATCACCATCAC
SEQ ID NO.: 24: DNA sequence of Light chain Artificial Human Antibody
comprising: Light Chain Variable Region and Light Chain Constant
Region
GATATCGAACTGACCCAGCCGCCGAGCGTGAGCGTGAGCCCGGGCCAGACCGCGAGCATTACCTGTAGCG
GCGATGCTCTGGGTTCTAAATACGTTCATTGGTACCAGCAGAAACCGGGCCAGGCGCCGGTGCTGGTGAT
CTACGCTAAAAACAACCGTCCGAGCGGCATCCCGGAACGTTTTAGCGGATCCAACAGCGGCAACACCGCG
ACCCTGACCATTAGCGGCACCCAGGCGGAAGACGAAGCGGATTATTACTGCCAGTCTCGTGCTTCTGGTA
TCTTCGGTCGTGTGTTTGGCGGCGGCACGAAGTTAACCGTTCTTGGCCAGCCGAAAGCCGCCCCAAGCGT
GACCCTGTTTCCGCCGAGCAGCGAAGAACTGCAAGCCAACAAAGCCACCCTGGTTTGCCTGATCAGCGAT
TTTTATCCGGGTGCCGTGACCGTGGCCTGGAAAGCCGATAGCAGCCCGGTGAAAGCCGGCGTGGAAACCA
CCACCCCGAGCAAACAGAGCAACAACAAATATGCCGCCAGCAGCTATCTGAGCCTGACCCCGGAACAGTG
GAAAAGCCATCGCAGCTATAGTTGTCAAGTGACCCATGAAGGCAGCACCGTGGAAAAAACCGTGGCCCCG
ACCGAGGCC

1. A method of detecting infection with Mycobacterium tuberculosis (M. tuberculosis) in a subject, said method comprising:
a) obtaining a test sample collected from the subject; and
b) detecting Mycobacteria modified Low Density Lipoprotein (MtLDL) in said test sample using at least one detection agent specific for MtLDL, wherein MtLDL in said test sample is indicative of infection with M. tuberculosis.

2. (canceled)

3. (canceled)

4. (canceled)

5. (canceled)

6. The method of claim 1, wherein an amount of MtLDL in a test sample of 4 ng/ml or more is indicative of infection with M. tuberculosis.

7. (canceled)

8. The method of claim 1, wherein the subject is infected with M. tuberculosis and Human Immunodeficiency virus (HIV).

9. (canceled)

10. The method of claim 1, wherein the test sample comprises blood, sputum, stool, urine, or cerebrospinal fluid (CSF).

11. (canceled)

12. (canceled)

13. (canceled)

14. (canceled)

15. The method of claim 1, wherein the test sample comprises stool.

16. The method of claim 1, wherein the detection agent binds MtLDL with greater than 5-fold specificity.

17. The method of claim 16, wherein the detection agent binds to at least one portion of the ApoB100 of MtLDL.

18. The method of claim 16, wherein the detection agent binds to at least one portion of MtLDL lipid.

19. (canceled)

20. (canceled)

21. The method of claim 1, wherein at least one detection agent is an antibody or antibody fragment thereof, said antibody or fragment comprising:
a) a heavy chain variable region (HCVR) comprising complementarity determining regions (CDRs) selected from the group consisting of: CDRs 1-3 of SEQ ID NO: 3, CDRs 1-3 of SEQ ID NO: 5, CDRs 1-3 of SEQ ID NO: 1, and CDRs 1-3 of SEQ ID NO: 7;
b) a light chain variable region (LCVR) comprising CDRs selected from the group consisting of: CDRs 1-3 of SEQ ID NO: 4; CDRs 1-3 of SEQ ID NO: 6, CDRs 1-3 of SEQ ID NO: 2, and CDRs 1-3 of SEQ ID NO: 8; or
c) both (a) and (b).

22. The method of claim 1, wherein at least one detection agent is an antibody or antibody fragment thereof, said antibody or fragment comprising:
a) a heavy chain variable region (HCVR) comprising an amino acid sequence selected from the group consisting of: SEQ ID NO: 3, SEQ ID NO: 5, SEQ ID NO: 1, and SEQ ID NO: 7;
b) a light chain variable region (LCVR) comprising an amino acid sequence selected from the group consisting of: SEQ ID NO: 4, SEQ ID NO: 6, SEQ ID NO: 2 and SEQ ID NO: 8; or
c) both (a) and (b).

23. (canceled)

24. (canceled)

25. (canceled)

26. The method of claim 1, wherein at least one detection agent is an antibody or antibody fragment thereof, said antibody or fragment detectably labeled with an enzyme or a fluorochrome or a recognition tag for a different antibody.

27. (canceled)

28. The method of claim 1, wherein the level of MtLDL in the test sample is detected using an enzyme-linked immunosorbent assay (ELISA).

29. (canceled)

30. A method for detecting the level of Mycobacteria modified Low Density Lipoprotein (MtLDL) in a test sample from a subject, said method comprising:
a) capturing MtLDL from the test sample on a solid phase with a first antibody or fragment thereof specific for an epitope of MtLDL, for a time and under conditions sufficient to form a MtLDL/first antibody complex;
b) separating the MtLDL/first antibody complex from free, unbound antigens in the test sample;
c) contacting the MtLDL/first antibody complex with a second antibody or fragment thereof, said second antibody or fragment specific for LDL, wherein the second antibody is specific for a different epitope of LDL than the first antibody, for a time and under conditions sufficient to form a MtLDL/first antibody/second antibody complex;
d) separating the MtLDL/first antibody/second antibody complex from free, unbound detection agents; and
e) detecting binding between the second antibody or antibody fragment and MtLDL using a labeled third antibody or antibody fragment or a labeled compound that recognizes the second antibody or antibody fragment but not the first antibody or antibody fragment.

31. The method of claim 30, wherein the second antibody is specific for MtLDL.

32. (canceled)

33. (canceled)

34. The method of claim 30, wherein the first antibody or fragment thereof binds to MtLDL but does not bind any other LDL species.

35. (canceled)

36. The method of claim 1, wherein at least one detection agent is an antibody or antibody fragment thereof, said antibody or fragment binding to the same epitope of MtLDL as an antibody or fragment thereof comprising the heavy chain variable region (HCVR) of SEQ ID NO: 3 and the light chain variable region (LCVR) of SEQ ID NO: 4, the HCVR of SEQ ID NO: 5 and the LCVR of SEQ ID NO: 6, the HCVR of SEQ ID NO: 1 and the LCVR of SEQ ID NO: 2, or the HCVR of SEQ ID NO: 7 and the LCVR of SEQ ID NO: 8.

37. (canceled)

38. The method of claim 1, wherein at least one detection agent is an antibody or antibody fragment thereof, said antibody or fragment comprising the heavy chain variable region (HCVR) of SEQ ID NO: 3 and the light chain variable region (LCVR) of SEQ ID NO: 4, the HCVR of SEQ ID NO: 5 and the LCVR of SEQ ID NO: 6, the HCVR of SEQ ID NO: 1 and the LCVR of SEQ ID NO: 2, or the HCVR of SEQ ID NO: 7 and the LCVR of SEQ ID NO: 8.

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. A kit suitable for detection of infection with M. tuberculosis in a sample, comprising:
a) at least one binding agent for binding specifically to and forming a complex with Mycobacteria modified Low Density Lipoprotein (MtLDL) in the sample;
b) a detectable signal-generating compound attached to the binding agent;
c) at least one separation reagent for separating the complex from free, unbound MtLDL and binding agents in the sample; and
d) a solid platform.

58. (canceled)

59. (canceled)

60. (canceled)

61. A method of treating infection with M. tuberculosis in a subject comprising:
a) obtaining a test sample collected from the subject;
b) detecting a level of Mycobacteria modified Low Density Lipoprotein (MtLDL) in the test sample using at least one detection agent specific for MtLDL,
c) diagnosing infection with M. tuberculosis if a level of MtLDL in the test sample is higher than a corresponding control sample; and
d) administering an appropriate treatment regimen to the subject upon diagnosis of infection with M. tuberculosis.

62. The method of claim 1, further comprising collecting the test sample from the subject.
